(19) United States
(12) Patent Application Publication
(10) Pub. No.: US 2025/0269182 A1
(43) Pub. Date: Aug. 28, 2025

(54) INTERMITTENT DUAL VAGUS NEUROMODULATION TREATMENT FOR IMPROVED GLYCEMIC CONTROL IN TYPE 2 DIABETES

(71) Applicant: RESHAPE LIFESCIENCES, INC., Irvine, CA (US)

(72) Inventors: Jonathan J. WAATAJA, Plymouth, MN (US); Rajesh K. NIHALANI, Irvine, CA (US)

(21) Appl. No.: 18/702,990

(22) PCT Filed: Oct. 24, 2022
(86) PCT No.: PCT/US2022/078598

§ 371 Date: Jul. 31, 2024

Related U.S. Application Data
(60) Provisional application No. 63/271,064, filed on Oct. 22, 2021.
(60) Provisional application No. 63/380,313, filed on Oct. 20, 2022.

(51) Int. Cl. A61N 1/36; A61N 1/05

(52) U.S. Cl. CPC A61N 1/36053; A61N 1/0551; A61N 1/3606; A61N 1/36139; A61N 1/36171

(57) ABSTRACT

A treatment method and system for the treatment and maintenance of patients with type 1 or type-2 diabetes. The variety of treatments described herein provides novel treatments that utilizes nerve stimulation alongside electrical blockade of a nerve branch to provide safe and effective treatment of type 1 and/or type-2 diabetes.

### CROSS-REFERENCE TO RELATED APPLICATION

This application is being filed on Oct. 24, 2022, as a PCT International Patent Application that claims priority to and the benefit of U.S. Application Ser. No. 63/271,064, filed Oct. 22, 2021, and U.S. Application Ser. No. 63/380,313 filed Oct. 20, 2022, the disclosures of which are hereby incorporated in its entirety.

### INTRODUCTION

Obesity is a major risk factor for the development of type 2 diabetes that affects over 31 million in the US. Compliance to type 2 diabetic medications and frequent glucose spikes continues to be a challenge in treating type 2 diabetes. Novel treatment options that are adjustable to patient's compliance and blunt glucose spikes are needed. More permanent treatments such as standalone vagotomy or vagus nerve stimulation have had undesirable or mixed results. Safety and reversibility of such treatments are also a significant concern. Bio-electronic neuromodulation of Vagus nerve branches innervating organs that regulate plasma glucose, may be a method for treating type 2 diabetes. There is a need to improve the treatments of type 2 diabetes.

### Intermittent Dual Vagus Neuromodulation Treatment for Improved Glycemic Control in Type 2 Diabetes

The present disclosure provides neuromodulation/neuroregulation systems and methods for treating type-2 diabetes. The invention described herein relates to a novel, adjustable and localized approach for a treatment of type 2 diabetes via electrical blockade of the hepatic Vagus branch with simultaneous electrical stimulation of the celiac Vagus branch. The Vagus nerve controls multiple organ systems that regulate blood glucose, such as the liver via the hepatic branch, and the pancreas via the celiac branch. There is mixed evidence that hepatic vagus nerve vagotomy may offer increased glycemic control. Nevertheless, there are inherent problems with nerve ligation: hepatic vagotomy is non-reversible, there may be adaptation to the vagotomy with time and peripheral nerves can regrow with unknown effects on the newly re-innervated end organ. Hepatic vagotomy may also cause negative changes in feeding behavior, increased hypoglycemic episodes, may affect liver regeneration and cause increased metastasis during liver cancer. Litigation is not the only option for inhibiting Vagal stimulation of the liver: introducing a reversible electrical blockade in the hepatic branch of the Vagus nerve have shown similar effects to litigation without the unwanted side-effects.

Previous research into electrical neuromodulation of the Vagus nerve for a treatment of type-2 diabetes have shown mixed results. Chronic studies using stimulation or electrical conduction blockade of the Vagus nerve have demonstrated increased glycemic control: however, weight loss may have been a contributing factor making it less suitable for many diabetics. Still other stimulation studies of the Vagus nerve, or its branches, have failed to increase glycemic control, with only a few studies demonstrating enhanced glucose regulation. The current invention combines stimulation of the celiac branch of Vagus nerve associated with the pancreas concurrently with the introduction of a reversible electrical blockade in the hepatic branch of the Vagus nerve. The current invention has shown significant results in animal models.

It should be noted that more recent optogenetic and chemo genetic vagal manipulation offers neuronal specificity and may be utilized for glycemic control, however, introducing a viral vector and genetic modulation presents as a real time clinical challenge in humans. Alternatively, electrical neuronal stimulation has a proven clinical safety and effective over the years in many therapeutic areas such as cervical stimulation for epilepsy, pain management, deep brain stimulation, urinary bladder control and other therapies. However, there is a need for electrical neuronal stimulation for the treatment and management of type-2 diabetes. In addition, the methods of systems described herein will also allow for artificial intelligence and machine learning to be utilized to optimize therapy settings & algorithms.

In some aspects, the present disclosure provides systems and methods for type-2 diabetes vagal nerve stimulation (T2D-VNS). In particular embodiments, the present T2D-VNS system comprises a pulse generator (PG), either implantable or external, in a closed loop with a continuous glucose monitor (CGM), stimulation electrodes/leads attachable to posterior vagus nerve (PVN) cranial to the celiac branch, a programmer to alter settings for therapeutic customization.

In some aspects, the present disclosure also provides a minimally invasive electrode implantation method. In particular embodiments, the present method includes implanting electrodes in a subject to be treated using a less invasive laparoscopic technique for optimal electrode placement with enhanced visualization of the posterior vagus nerve and celiac branch. This can be achieved by reliably locating the celiac branch laparoscopically for correct electrode placement on the PVN.

In some aspects, the present disclosure provides various operating parameters for T2D-VNS. In particular embodiments, implementation of the present method using selected operating parameters is effective to increase plasma glucose by at least about 20 mg/dL within about 30 min after treatment in a subject from a controlled clamped glucose level of 50 mg/dL.

In some aspects, the present disclosure provides the safety of stimulation on vagal nerve and end organs. From animal studies presented in the Examples of this disclosure, little-to-no adverse behavior or organ damage is observed as a result of stimulation or gross necropsy.

In some aspects, a system for treating type-2 diabetes in a subject comprises: (1) at least one electrode adapted to be placed on and deliver electrical signal to a posterior vagus nerve (PVN) of the subject or the celiac vagus nerve branch of the PVN: (2) a pulse generator operably connected to the at least one electrode, wherein the pulse generator comprises a power module and a programmable therapy delivery module, wherein the programmable therapy delivery module is configured to deliver at least one therapy program, wherein the at least one therapy program comprises at least one electrical signal treatment applied to the PVN through the at least one electrode, (3) an external component comprising a communication system and a programmable storage and communication module, wherein programmable storage and communication module are configured to store the at least one therapy program and to communicate the at least one therapy program to the pulse generator, and (4) a glucose sensor operably connected to the pulse generator and the external component, wherein the glucose sensor is configured to continuously monitor plasma glucose of the subject and to detect an increase or decrease of plasma glucose from a pre-determined threshold level, wherein, the pulse generator is triggered to deliver the at least one electrical signal treatment when the plasma glucose of the subject is of or below a first pre-determined threshold, wherein the pulse generator ceases to deliver the at least one electrical signal treatment when the plasma glucose of the subject is of or above a second pre-determined threshold, wherein the at least one electrical signal treatment comprises an electrical signal pattern having a frequency from about 1 Hz to about 200 Hz, a pulse width from about 0.1 microseconds (ms) to about 10 ms in about 0.1 ms steps, a pulse amplitude from about 0.1 mA to about 12 mA in about 0.1 mA steps, and wherein the electrical signal treatment is configured to initiate neural stimulation on PVN of the subject.

In some embodiments, a method of treating type-2 diabetes in a subject comprises: applying the at least one electrical signal treatment to a posterior vagus nerve (PVN) of a subject or the celiac vagus nerve branch of the PVN of the subject using the present system.

In another example, a system for treating type-2 diabetes in a subject comprises: (1) a first electrode adapted to be placed on and deliver electrical signal to a first nerve or organ: (2) optionally a second electrode adapted to be placed on and deliver electrical signal to a second nerve or organ: (3) an pulse generator operably connected to the first and/or the second electrode, wherein the pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and (4) an external component comprising a communication system and a programmable storage and communication module, wherein programmable storage and communication module are configured to store the at least one therapy program and to communicate the at least one therapy program to the pulse generator.

In some embodiments, a method of treating type-2 diabetes in a subject, the method comprising: (1) applying a first electrical signal to a first nerve or organ of the subject using the system of claim 1, wherein the first electrical signal initiates a neural stimulation or a neural block; and (2) optionally applying a second electrical signal to a second nerve or organ of the subject using the system of claim 1, wherein the second electrical signal initiates a neural stimulation or a neural block.

In some embodiments, the first and/or the second electrical signal are each independently configured to upregulate or downregulate activity respectively on the first and/or second target nerve or organ. In some embodiments, the first and the second electrical signals are applied concurrently, or simultaneously, or intermittently, or during substantially the same times, or during substantially different times, or in a coordinated fashion. In some embodiments, the first and/or the second electrical signal treatments are each continuously applied to the first target nerve or organ and/or the second target nerve or organ respectively. In certain embodiments, the first electrical signal is an upregulation or stimulation signal.

In some embodiments, the method further comprises a glucose sensor configured to continuously monitor plasma glucose of the subject, wherein the glucose sensor is operably connected to the pulse generator and the external component. In some embodiments, the glucose sensor is configured to detect an increase or decrease of plasma glucose from a pre-determined threshold level. In some embodiments, the pulse generator is triggered to deliver the first and/or the second electrical treatment when the plasma glucose of the subject is of or below a first pre-determined threshold, and wherein the pulse generator ceases to deliver the first and/or the second electrical treatment when the plasma glucose of the subject is of or above a second pre-determined threshold.

In some embodiments, the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof. In particular embodiments, the first nerve or organ is celiac branch of posterior vagus nerve.

In some embodiments, the first nerve or organ and the second nerve or organ are different. In some embodiments, the first electrical signal is applied on a hepatic branch of a vagus nerve or a ventral vagus nerve central to a branching point of a hepatic nerve. In some embodiments, the first electrical signal is applied on a celiac branch of a vagus nerve, or a ventral vagus nerve central to a branching point of a celiac nerve, or liver, pancreas, or both.

In some embodiments, the first and/or the second electrical signals each have an on time and an off time, wherein the off time is selected to allow at least a partial recovery of the activity of the first and/or the second nerve or organ. In some embodiments, the on time is configured to commence upon the detection of plasma glucose level of 50 mg/dL, or about 60 mg/dL, or about 70 mg/dL, or about 80 mg/dL. In some embodiments, the first and/or the second electrical signal treatment are configured to alter the plasma glucose level by at least about 5 mg/dL in about 10 minutes. In some embodiments, the first and/or the second electrical signal treatment are configured to alter the plasma glucose level by at least about 10 mg/dL in about 20 minutes. In some embodiments, the first and/or the second electrical signal treatment are configured to alter the plasma glucose level by at least about 20 mg/dL in about 30 minutes.

In some embodiments, the first electrical signal has a frequency of about 0.01 Hz to about 200 Hz, from about 0.1 Hz to about 100 Hz, or from about 1 Hz to about 20 Hz. In other embodiments, the first electrical signal has a frequency of about 200 Hz to about 10 k Hz.

In some embodiments, the second electrical signal has a frequency of about 0.01 Hz to about 200 Hz, from about 0.1 Hz to about 100 Hz, or from about 1 Hz to about 20 Hz. In other embodiments, the second electrical signal has a frequency of about 200 Hz to about 10 k Hz.

In some embodiments, the first electrical signal has a frequency of about 0.01 Hz to about 200 Hz, and wherein the second electrical signal has a frequency of about 200 Hz to about 10 k Hz.

In some embodiments, the first electrical signal and/or the second electrical signal each independently comprise a signal pattern, wherein each signal pattern comprises a pulse having a pulse width from about 10 microseconds to about 10,000 microseconds.

In some embodiments, the pulse of the first and/or the second electrical signal is monophasic pulse, or biphasic pulse, or combinations thereof. In some embodiments, the first and/or the second electrical signal each independently have an on time of about 30 seconds to about 30 minutes. In some embodiments, the first and/or the second electrical signal each independently have a current amplitude in a range from about 0.01 mAmps to about 20 mAmps. In some embodiments, the first and/or the second electrical signal each independently comprise an abrupt start of pulses, or a ramp up of current/voltage amplitude, or a ramp up of frequency, or a ramping up of pulse widths, or combination thereof at or near initiation of applying the first and/or the second electrical signal.

In some embodiments, the first and/or the second electrical signal treatments are configured to be applied intermittently multiple times in a day and/or over multiple days, wherein the first and/or the second electrical signal each have a frequency selected to upregulate activity (or downregulate) on the first nerve or organ and has an on time and/or an off time, wherein the off time is selected to allow at least a partial recovery of the activity of the first or second nerve or organ.

In some embodiments, the first and/or the second electrical signal treatments are configured to be applied intermittently multiple times in a day and over multiple days, wherein the first and/or the second electrical signal each have a frequency selected to upregulate activity on the first nerve or organ and has an on time and an off time, wherein the off time is selected to allow at least a partial recovery of the activity of the first nerve or organ.

In some embodiments, the programmable storage and communication module are configured to store and communicate more than one therapy program, wherein each therapy program is different from one another, and is configured to be selected for communication.

In some embodiments, the system further comprises a transmitter operably connected to the glucose sensor, wherein the transmitter is configured to communicate data generated by the glucose sensor to an external communication device.

In some embodiments, the communication system is selected from a group consisting of an antenna, blue tooth technology, radio frequency, WIFI, light, sound and combinations thereof, and wherein the communication system is configured to communicate parameters of the at least one therapy program to an external communication device.

In some embodiments, a method of making a system for treating type-2 diabetes in a subject comprises: (1) connecting a first electrode to an pulse generator and placing the first electrode to a first nerve or organ: (2) optionally connecting a second electrode to the pulse generator and placing the second electrode to a second nerve or organ: (3) configuring a programmable therapy delivery module of the pulse generator to deliver at least one therapy program comprising a first electrical signal treatment and optionally a second electrical signal treatment, wherein the first electrical signal treatment is configured to be applied to the first nerve or organ through the first electrode, and the second electrical signal treatment is configured to be applied to the second nerve or organ through the second electrode, and wherein the first and/or the second electrical signal each initiate a neural stimulation or a neural block; and (4) configuring a programmable storage and communication module of an external component to store the at least one therapy program and to communicate the at least one therapy program to the pulse generator.

### Definition and Interpretation of Selected Terms

The term “about” is not intended to either expand or limit the degree of equivalents which may otherwise be afforded a particular value. The term “about” in the context of the present disclosure means a value within 10% (+10%) of the value recited immediately after the term “about,” including any numeric value within this range, the value equal to the upper limit (i.e., +10%) and the value equal to the lower limit (i.e., −10%) of this range. For example, the value “100” encompasses any numeric value that is between 90 and 110, including 90 and 110 (with the exception of “100%,” which always has an upper limit of 100%).

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

“Cycle” as used herein means one repetition of a repetitive pattern of electrical signals. “Stimulation cycle” particularly refers to low frequency stimulation signal. “Concurrently” used here in generally means that in situations where multiple electrical signals are applied, in at least one time period, the multiple electrical signals are applied simultaneously or about the same time.

“Duty Cycle” as used herein means the percentage of time charge is delivered to the nerve in one cycle. In embodiments, duty cycle can be modified by decreasing pulse width and/or by adding inactive phases between pulses or both.

“Frequency” as used herein means the reciprocal of the period measured in

### Hertz.

“High Duty Cycle” as used herein refers to a pattern of electrical signals with a duty cycle of about 76% or greater.

“Low Duty Cycle” as used herein refers to a pattern of signals with a duty cycle of about 75% or less.

“High frequency” as used herein generally refers to a frequency of about 200 Hz or more. “High frequency signal” as used herein generally refers to HFAC or HFAV having a frequency of about 200 Hz or more. High frequency signal is particularly used to downregulate or block nerve activity.

“Low frequency” as used herein generally refers to a frequency of about 200 Hz or less.

“Low frequency signal” or “low frequency stimulation signal” as used herein generally refers to stimulation signal having a frequency of 199 Hz or less. Stimulation signal is particularly used to upregulate or stimulate nerve activity.

“HFAC” as used herein refers to high frequency alternating current.

“HFAV” as used herein refers to high frequency alternating voltage.

“Hz” as used herein refers to Hertz.

“Intermittent” as used herein refers to a treatment duration that does not exceed 60 minutes, more preferably does not exceed 45 minutes, even more preferably does not exceed 30 minutes.

“Off Time” as used herein refers to a period when no charge is being delivered to the nerve. In embodiments, off time is on the order of seconds and/or minutes.

“On Time” refers to a period of time in which multiple micro and/or millisecond cycles and/or stimulation cycle and/or stimulation active phase are applied to the nerve. In embodiments, on time is on the order of seconds and/or minutes.

“Period” refers to the length of time of one charge phase and one recharge phase, which can include one or more pulse delays. “Stimulation period” particularly refers to the length of time of one charge phase and one recharge phase in a low frequency stimulation signal. Stimulation period can also include one or more pulse delays.

“Pulse Amplitude” is the height of the pulse in amperes or voltage relative to the baseline.

“Pulse Delay” as used herein refers to an aspect of the period wherein the impedance across a parallel electrical path with the nerve is at or close to 0 Ohms, with the intention of avoiding any unwanted electrical signals being delivered to the nerve.

“Pulse Width” as used herein refers to the length of time of the pulse.

“Ramp Down” as used herein refers to the period at the end of the application of an electrical signal, or between different patterns of electrical signals, to a nerve of a patient where the pulse amplitude of the signal decreases.

“Ramp Up” as used herein refers to increasing the pulse amplitude until the amplitude desired for therapy is reached at the start of an applied electrical signal or between different patterns of electrical signals. The starting amplitude of ramping may be below the current/voltage threshold of blocking.

“Therapy Cycle” as used herein refers to a discrete period of time that contains one or more on times and off times. The pattern of on and off times within the therapy cycle can be repetitive, non-fixed or randomized throughout a therapy schedule.

“Therapy Parameters” as used herein includes, but is not limited to, frequency, pulse width, pulse amplitude, on time, off time and pattern of electrical signals.

“Therapy Schedule” as used herein refers to the time of day when therapy cycles start, the number of therapy cycles, timing of therapy cycles and duration of the delivery of therapy cycles for at least one day of the week.

“Nerve” used herein generally encompasses a nerve or any part thereof, including but not limited to nerve branch, nerve fiber, trunk, branching point.

“Anterior vagus nerve (AVN)” or “anterior vagus trunk” distributes fibers on the anterior surface of the esophagus, and consists primarily of fibers from the left vagus. “Posterior vagus nerve (PVN)” or “posterior vagus trunk” consists primarily of fibers from the right vagal nerve distributed on the posterior surface of the esophagus. Anterior vagus nerve and posterior vagus nerve are two different and separate nerves.

“Hepatic branch” used herein refers to a nerve branch of the anterior vagus nerve below the diaphragm. Hepatic branch encompasses any segment of the anterior vagus nerve cranial to the hepatic branch. In particular, Hepatic branch carries afferent information from the pancreas to the brain and efferent information from the brain to the pancreas.

“Celiac branch” used herein generally refers to a nerve branch of the posterior vagus nerve below the diaphragm. Celiac branch encompasses any segment of the posterior vagus nerve cranial to celiac branch. In particular, celiac branch carries afferent information from the pancreas to the brain and efferent information from the brain to the pancreas.

“Celiac fiber” used herein refers to an afferent or efferent axon that travels within the length of the vagal nerve between the pancreas and the brain. The afferent axon travels from the pancreas through the celiac branch of the vagal nerve where it then travels into the posterior vagus below the level of the diaphragm. The afferent axon next enters the thoracic cavity and primarily into the right cervical segment. The afferent axon then enters the brainstem and form a synaptic connection. The efferent fiber is a part of the parasympathetic nervous system. The preganglionic cell body of the efferent fiber is in the brain stem and travels the length of the vagal nerve (similar to the afferent fiber) to its postganglionic neuron in close proximity to the pancreas.

“Hepatic fiber” used herein refers to an afferent or efferent axon that travels within the length of the vagal nerve between the liver and the brain. The afferent axon travels from the liver through the hepatic branch of the vagal nerve where it then travels into the anterior vagus below the level of the diaphragm. The afferent axon next enters the thoracic cavity and primarily into the left cervical segment. The afferent axon then enters the brainstem and form a synaptic connection. The efferent fiber is a part of the parasympathetic nervous system. The preganglionic cell body of the efferent fiber is in the brain stem and travels the length of the vagal nerve (similar to the afferent fiber) to its postganglionic neuron in close proximity to the liver.

When ranges are provided, the range includes both endpoint numbers as well as all real numbers in between. For example, a range of 200 Hz to 25 kHz includes, for example, 201 to 25 kHz, 202 to 25 kHz, as well as 24,999 Hz to 200 Hz, 24,998 Hz to 200 Hz, and 201 Hz to 24,999 Hz, 202 Hz to 24,998 Hz.

With reference now to the various drawing figures in which identical elements are numbered identically throughout, a description of embodiments of the present disclosure will now be described.

### BRIEF DESCRIPTION OF THE DRAWINGS

The following images are incorporated in and constitute a part of the description, illustrating several aspects of the present invention. A brief description of the Figures is as follows:

FIG. 1 depicts the Vagus nerve, relevant pathways and associated organs.

FIG. 2 depicts the location of the blocking and stimulation electrodes.

FIG. 3. illustrates a schematic representation of an exemplary system comprising a pulse generator and leads comprising electrodes placed on an anterior vagus nerve (AVN) and posterior vagus nerve (PVN).

FIG. 4 illustrates a schematic representative of another exemplary system, in accordance with various embodiments of the present disclosure.

FIG. 5 illustrates an exemplary architecture of a computing device that can be used to implement aspects of the present disclosure.

FIG. 6 is a flowchart illustrating an exemplary method of operating the present system.

FIG. 7 illustrates another exemplary HVNS system in a disassembled configuration with individual components thereof, according to Example 1.

FIG. 8 shows a schematic of system in which an implantable glucose sensor communicates with a pulse generator to initiate vagus nerve stimulation.

FIG. 9 shows a schematic of system in which an implantable glucose sensor communicates first with an external device attached to the outside of the skin which then communicates with the pulse generator to initiate vagus nerve stimulation.

FIG. 10 shows the blocking and stimulation signals at 5000 Hz and 1 Hz square waves, respectively.

FIG. 11 illustrates the change of plasma glucose in rats as a result of the disclosed treatment compared to sham treatment during and after the administration of intravenous glucose in rats.

FIG. 12 illustrates the change of plasma glucose in rats as a result of the disclosed treatment compared to experimental treatments.

FIG. 13 illustrates a representative compound action potential elicited by electrical stimulation below the level of the diaphragm.

FIG. 14 illustrates a strength-duration curve indicating stimulus threshold for vagus nerve CAPs. Curve is fit with a power function.

FIG. 15 compares the current-effect curve of CAP amplitude recorded immediately following the cessation of 5000 Hz at a proximal and distal location on the nerve.

FIG. 16 demonstrates recovery of CAP amplitude following full block at 8 mA.

FIG. 17 illustrates blood glucose levels after injection of intravenous glucose pre- and post-application of Alloxan.

FIG. 18 illustrates insulin response at the 3 minutes after injection of intravenous glucose pre- and post-application of Alloxan.

FIG. 19 illustrates the change in plasma glucose in swine before and after implant of electrodes.

FIG. 20 illustrates the change of plasma glucose in swine as a result of the disclosed treatment compared to sham control.

FIG. 21 illustrates high frequency alternating current in Alloxan treated swine.

FIG. 22 illustrates fluctuation in plasma glucose in swine receiving the disclosed treatment compared to swine receiving sham treatment.

FIG. 23 illustrates the plasma glucose in swine receiving the disclosed treatment for extended periods compared to those receiving intermittent or sham treatments.

FIG. 24 depicts the histopathology of various tissues post application of the present invention.

### DETAILED DESCRIPTION

### Therapy System

In some aspects, the present disclosure provides systems and devices for treating a condition associated with type-2 diabetes. The system generally includes a pulse generator that provides signals to modulate neural activity on a target nerve or organ.

In some embodiments, a system according to the present disclosure comprises at least one electrode operably connected to an implantable pulse generator, wherein the electrode is adapted to be placed on a target nerve and/or a target organ of a subject: an implantable pulse generator that comprises a power module and a programmable therapy delivery module, wherein the programmable therapy delivery module is configured to deliver at least one therapy program comprising an electrical signal treatment applied intermittently multiple times in a day and over multiple days to the target nerve, wherein the electrical signal has a frequency selected to upregulate (for neural stimulation) nerve activity and/or downregulate (for neural block) on the target nerve and/or organ and has an on time and an off time, wherein the off time is selected to allow at least a partial recovery of the activity of the target nerve or organ; and an external component comprising an antenna and a programmable storage and communication module, wherein programmable storage and communication module is configured to store the at least one therapy program and to communicate the at least one therapy program to the implantable pulse generator.

In some embodiments, the system may include two electrodes, e.g., a first electrode and a second electrode, each operably connected to the implantable pulse generator. The first electrode is adapted to be placed on and deliver electrical signal to a first nerve or organ of the subject. The second electrode is adapted to be placed on and deliver electrical signal to a second nerve or organ of the subject. The system may comprise at least two therapy program, e.g., a first therapy program and optionally a second therapy program. The first therapy program comprises a first electrical signal treatment applied to the first nerve or organ through the first electrode, and the second therapy program comprises a second electrical signal treatment applied to the second nerve or organ through the second electrode. The first and/or the second electrical signal are each configured to initiate activity on the first and/or the second nerve or organ respectively, and wherein the activity is a neural stimulation or a neural block.

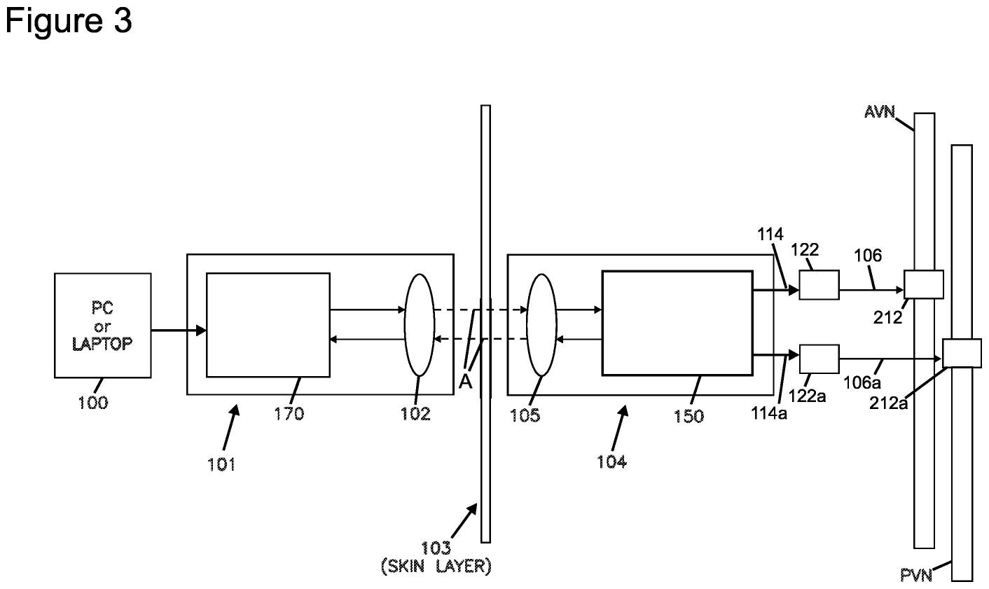

Now referring to FIG. 3, one example system according to the present disclosure and various aspects thereof will be described. In the illustrated example, a system for treating type-2 diabetes or a condition associated with type-2 diabetes includes a pulse generator 104, an external mobile charger 101, and two electrical lead assemblies 106, 106a. The pulse generator 104 is adapted for implantation within a subject to be treated. In some embodiments, the pulse generator 104 is implanted just beneath a skin layer 103 of the subject. In related embodiments the system includes 1 or more pulse generators 104.

In some embodiments, the lead assemblies 106, 106a are electrically connected to the circuitry of the pulse generator 104 by conductors 114, 114a. Industry standard connectors 122, 122a are provided for connecting the lead assemblies 106, 106a to the conductors 114, 114a. As a result, leads 116, 116a and the pulse generator 104 may be separately implanted. Also, following implantation, lead 116, 116a may be left in place while the originally placed pulse generator 104 is replaced by a different pulse generator.

The lead assemblies 106, 106a upregulate and/or downregulate nerves of the subject based on the therapy signals provided by the neuroregulator 104. In one embodiment, the lead assemblies 106, 106a include distal electrodes 212, 212a, which are placed on one or more target nerves or target organs of the subject. For example, the electrodes 212, 212a may be individually placed on the celiac nerve, the vagal nerve, the celiac branches of the vagal nerve, the hepatic branches of the vagal nerve, or some combination of these, respectively, of the subject to be treated. For example, the leads 106, 106a have distal electrodes 212, 212a which are individually placed on the PVN and AVN, respectively, of the subject, for example, just below the patient's diaphragm. Fewer or more electrodes can be placed on or near fewer or more nerves. In some embodiments, only one electrode is placed on the PVN of the subject, and no more electrode is placed on any other nerve or organ of the same subject. In some embodiments, the electrodes are cuff electrodes.

The external mobile charger 101 includes circuitry for communicating with the implanted neuroregulator (pulse generator) 104. In some embodiments, the communication is a two-way radiofrequency (RF) signal path across the skin 103 as indicated by arrows A. Example communication signals transmitted between the external charger 101 and the neuroregulator 104 include treatment instructions, patient data, and other signals as will be described herein. Energy or power also can be transmitted from the external charger 101 to the neuroregulator 104 as will be described herein.

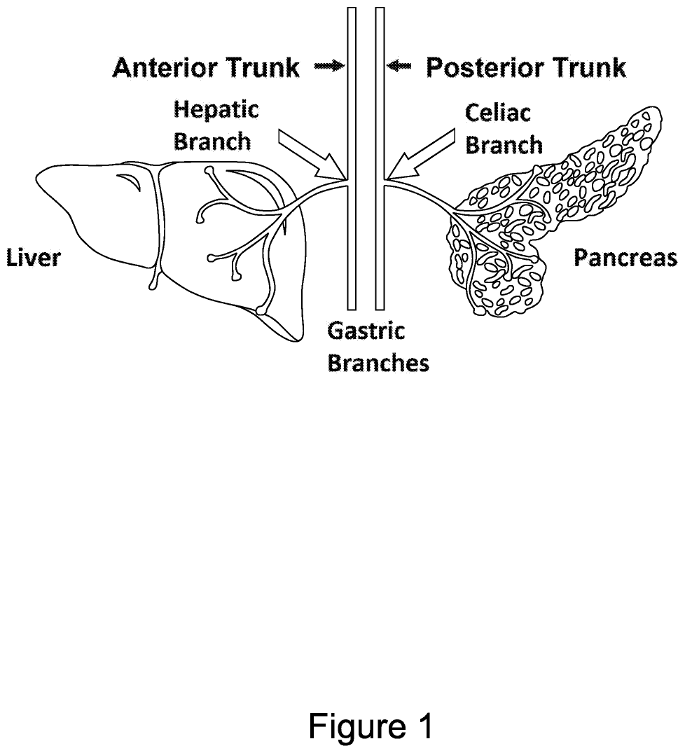

In the example shown, the external charger 101 can communicate with the implanted neuroregulator 104 via bidirectional telemetry (e.g. via radiofrequency (RF) signals). The external charger 101 shown in FIG. 1 includes a coil 102, which can send and receive RF signals. A similar coil 105 can be implanted within the patient and coupled to the neuroregulator 104. In an embodiment, the coil 105 is integral with the neuroregulator 104. The coil 105 serves to receive and transmit signals from and to the coil 102 of the external charger 101.

For example, the external charger 101 can encode the information as a bit stream by amplitude modulating or frequency modulating an RF carrier wave. The signals transmitted between the coils 102, 105 preferably have a carrier frequency of about 6.78 MHz. For example, during an information communication phase, the value of a parameter can be transmitted by toggling a rectification level between half-wave rectification and no rectification. In other embodiments, however, higher or lower carrier wave frequencies may be used.

In one embodiment, the neuroregulator 104 communicates with the external charger 101 using load shifting (e.g., modification of the load induced on the external charger 101). This change in the load can be sensed by the inductively coupled external charger 101. In other embodiments, however, the neuroregulator 104 and external charger 101 can communicate using other types of signals.

In an embodiment, the neuroregulator 104 receives power to generate the therapy signals from an implantable power source 151 such as a battery. In a preferred embodiment, the power source 151 is a rechargeable battery. In some embodiments, the power source 151 can provide power to the implanted neuroregulator 104 when the external charger 101 is not connected. In other embodiments, the external charger 101 also can be configured to provide for periodic recharging of the internal power source 151 of the neuroregulator 104. In an alternative embodiment, however, the neuroregulator 104 can entirely depend upon power received from an external source. For example, the external charger 101 can transmit power to the neuroregulator 104 via the RF link (e.g., between coils 102, 105). In a further embodiment, charging of the rechargeable battery 151 in the neuroregulator 104, can be achieved by application of remote wireless energy. (Grajski et al, IEEE Microwave Workshop series on Innovative Wireless Power Transmission: Technology, Systems, and Applications, 2012 published on a4wp.org).

In some embodiments, the neuroregulator 104 initiates the generation and transmission of therapy signals to the lead assemblies 106, 106a. In an embodiment, the neuroregulator 104 initiates therapy when powered by the internal battery 151. In other embodiments, however, the external charger 101 triggers the neuroregulator 104 to begin generating therapy signals. After receiving initiation signals from the external charger 101, the neuroregulator 104 generates the therapy signals (e.g., pacing signals) and transmits the therapy signals to the lead assemblies 106, 106a.

In other embodiments, the external charger 101 also can provide the instructions according to which the therapy signals are generated (e.g., pulse-width, amplitude, frequency, wave phase, and other such parameters). In some embodiments, the external component comprises an communication system and a programmable storage and communication module. Instructions for one or more therapy programs can be stored in the programmable storage and communication module. In a preferred embodiment, the external charger 101 includes memory in which several predetermined programs/therapy schedules can be stored for transmission to the neuroregulator 104. The external charger 101 also can enable a user to select a therapy program/therapy schedule stored in memory for transmission to the neuroregulator 104. In another embodiment, the external charger 101 can provide treatment instructions with each initiation signal.

Typically, each of the therapy programs/therapy schedules stored on the external charger 101 can be adjusted by an operator (such as a physician) to suit the individual needs of the subject (e.g., a patient to be treated). For example, a computing device (e.g., a notebook computer, a personal computer, etc.) 100 can be communicatively connected to the external charger 101. With such a connection established, an operator can use the computing device 107 to program therapies into the external charger 101 for either storage or transmission to the neuroregulator 104.

The neuroregulator 104 also may include memory in which treatment instructions and/or patient data can be stored. In some embodiments, the neuroregulator comprises a power module and a programmable therapy delivery module. For example, the neuroregulator 104 can store one or more therapy programs in the programmable therapy delivery module indicating what therapy should be delivered to the subject. The neuroregulator 104 also can store therapy/treatment/patient data indicating how the patient utilized the therapy system and/or reacted to the delivered therapy.

In some embodiments, the external component and/or the neuroregulator, are programmed with one or more therapy programs. One therapy program may comprise an electrical signal treatment applied intermittently multiple times in a day and over multiple days, wherein the electrical signal has a frequency selected to downregulate and/or upregulate activity on a first target nerve and has an on time and an off time, wherein the off time is selected to allow at least a partial recovery of the activity of the first target nerve. Another therapy program may comprise an electrical signal treatment applied continuously over multiple days, wherein the electrical signal has a frequency selected to downregulate and/or upregulate activity on the first target nerve or organ. A second therapy program may comprise an electrical signal treatment applied intermittently multiple times in a day and over multiple days, wherein the electrical signal has a frequency selected to upregulate or downregulate activity on a second target nerve or organ, and has an on time and an off time, wherein the off time is selected to allow at least a partial recovery of the activity of the second target nerve. The first and/or second therapy programs may be applied at the same time, at different times, or at overlapping times. The first and/or second therapy programs may be delivered at specific times of the day, and or in response to a signal from a sensor. In some embodiments the sensor is designed to measure the plasma glucose level of a patient. In some embodiments the off time is configured to commence upon the detection of plasma glucose levels between 50 mg/dL and 90 mg/dL. In some embodiment the on time is configured to commence upon the detection of plasma glucose levels below 90 mg/dL, below 80 mg/dL, below 70 mg/dL, below 60 mg/dL, or below: 50 mg/dL.

In some embodiments, the present system further comprises a biological sensor (not shown). The biological sensor may be an independent unit integrated into the therapy system, or be otherwise operatively coupled to the system. In embodiments, the biological sensor is electrically connected to the system. In embodiments, the biological sensor is in wireless communication with the therapy system. In embodiments, the biological sensor is operatively coupled to the neuroregulator of the therapy system. For example, a sensing electrode SE of the biological sensor can be added to monitor neural activity as a way to determine how to modulate the neural activity and/or the duty cycle. While sensing electrode can be an additional electrode to blocking electrode, it will be appreciated a single electrode could perform both functions. The sensing and blocking electrodes can be connected to a controller as shown in FIG. 1. Such a controller is the same as controller 102 previously described with the additive function of receiving a signal from sensing electrode.

In some embodiments, the sensor can be a sensing electrode, a glucose sensor, or sensor that senses other biological molecules or hormones of interest. When the sensing electrode SE yields a signal representing a targeted maximum vagal activity or tone, the controller with the additive function of receiving a signal from sensing electrode functions to change and/or maintain the signals delivered to the electrode(s) placed on nerve branches/fibers. As described with reference to controller 102, controller with the additive function of receiving a signal from sensing electrode can be remotely programmed as to parameters of blocking/stimulating duration and no blocking/stimulation duration as well as targets for initiating, or maintaining, or ceasing, or terminating, or otherwise manipulating the blocking signal and/or upregulating signal.

In practicing the therapy system, depending upon the glucose value of the subject indicated by the glucose sensor, the system can apply responsive changes to the first and/or the second electrical signal to control/maintain the plasma glucose at a demanded level.

In one particular embodiment, system 100 includes a glucose sensor configured to continuously monitor plasma glucose of the subject, wherein the glucose sensor is operably connected to the implantable pulse generator 104 and the external component. The glucose sensor is configured to detect an increase or decrease of plasma glucose from a pre-determined threshold level. The implantable pulse generator 104 is triggered to deliver the first and/or the second electrical treatment when the plasma glucose of the subject is of or below a first pre-determined threshold, and wherein the implantable pulse generator ceases to deliver the first and/or the second electrical treatment when the plasma glucose of the subject is of or above a second pre-determined threshold. The pre-determined threshold of the plasma glucose is about 50 mg/dL and 90 mg/dL. In some embodiment the on time is configured to commence upon the detection of plasma glucose levels below 90 mg/dL, below 80 mg/dL, below 70 mg/dL, below 60 mg/dL, or below: 50 mg/dL.

The circuitry 170 of the external mobile charger 101 can be connected to an external coil 102. The coil 102 communicates with a similar coil 105 implanted within the subject and connected to the circuitry 150 of the pulse generator 104. Communication between the external mobile charger 101 and the pulse generator 104 includes transmission of pacing parameters and other signals as will be described.

Having been programmed by signals from the external mobile charger 101, the pulse generator 104 generates upregulating signals and/or downregulating signals to the leads 106, 106a. As will be described, the external mobile charger 101 may have additional functions in that it may provide for periodic recharging of batteries within the pulse generator 104, and also allow record keeping and monitoring.

While an implantable (rechargeable) power source for the pulse generator 104 is preferred, an alternative design could utilize an external source of power, the power being transmitted to an implanted module via the RF link (i.e., between coils 102, 105). In this alternative configuration, while powered externally, the source of the specific electrical signals could originate either in the external power source unit, or in the implanted module.

The electronic energization package may, if desired, be primarily external to the body. An RF power device can provide the necessary energy level. The implanted components could be limited to the lead/electrode assembly, a coil and a DC rectifier. With such an arrangement, pulses programmed with the desired parameters are transmitted through the skin with an RF carrier, and the signal is thereafter rectified to regenerate a pulsed signal for application as the stimulus to the vagal nerve to modulate vagal activity. This would virtually eliminate the need for battery changes.

However, the external transmitter must be carried on the subject (e.g., the person of the patient), which is inconvenient. Also, detection is more difficult with a simple rectification system, and greater power is required for activation than if the system were totally implanted. In any event, a totally implanted system is expected to exhibit a relatively long service lifetime, amounting potentially to several years, because of the relatively small power requirements for most treatment applications. Also, as noted earlier herein, it is possible, although considerably less desirable, to employ an external pulse generator with leads extending percutaneously to the implanted nerve electrode set. The major problem encountered with the latter technique is the potential for infection. Its advantage is that the patient can undergo a relatively simple procedure to allow short term tests to determine whether the condition associated with excess weight of this particular patient is amenable to successful treatment. If it is, a more permanent implant may be provided.

In some embodiments, the present system is configured to apply an electrical signal to an internal anatomical feature of a subject. The system includes at least one electrode for implantation within the subject and placement at the anatomical feature (e.g., a nerve) for applying the signal to the feature upon application of the signal to the electrode. An implantable component is placed in the subject's body beneath a skin layer and having an implanted circuit connected to the electrode. The implanted circuit includes an implanted communication system. An external component has an external circuit with an external communication system for placement above the skin and adapted to be electrically coupled to the implanted communication system across the skin through radiofrequency transmission. The external circuit has a plurality of user interfaces including an information interface for providing information to a user and an input interface for receiving inputs from the user.

In some embodiments, the present system is configured to apply electrical signals to different vagal nerve branches. For example, the esophagus passes through the diaphragm at an opening or hiatus. In the region where the esophagus passes through the diaphragm, trunks of the vagal nerve (e.g., AVN or PVN) are disposed on opposite sides of the esophagus. It will be appreciated that the precise location of the AVN and PVN relative to one another and to the esophagus are subject to a wide degree of variation within a patient population. However, for many subjects, the AVN and PVN are in close proximity to the esophagus at the hiatus where the esophagus passes through the diaphragm. The AVN and PVN may divide into a plurality of trunks that innervate organs such as the pancreas, gallbladder, liver, stomach, and intestines. Commonly, the AVN and PVN are still in close proximity to the esophagus and stomach (and not yet extensively branched out) at the region of the junction of the esophagus and stomach.

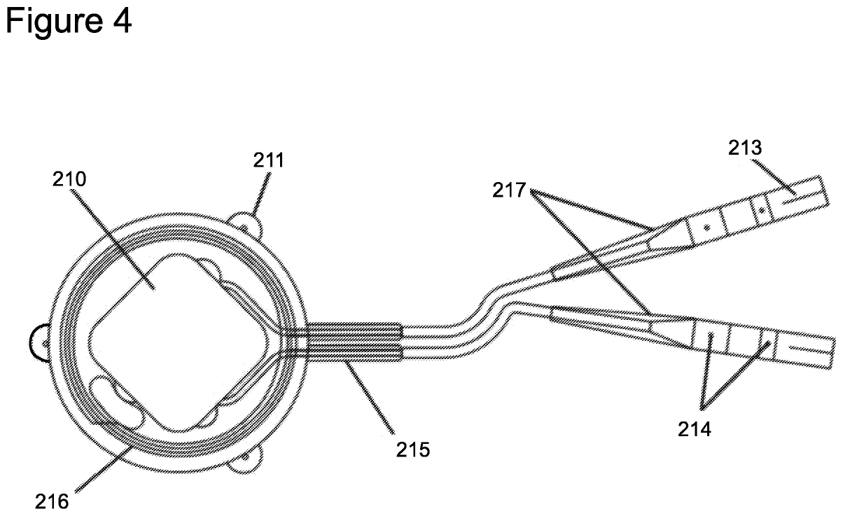

Now referring to FIG. 4, another example of the present system useful in treating a condition associated with type-2 diabetes will be illustrated and described. With reference to FIG. 4, a device comprises an implantable component comprising an electronic assembly 210 (“hybrid circuit”) and a receiving coil 216: standard connectors 217 (e.g. IS-1 connectors) for attachment to electrode leads. Two leads are connected to the IS-1 connectors for connection to the implanted circuit. Both have a tip electrode for placement on a nerve. Set screws are shown in 214 and allow for adjustment of the placement of the electrodes. In some embodiments, a marker 213 to indicate the dorsal or ventral lead is provided. Suture tabs 211 are provided to provide for implantation at a suitable site. In some embodiments, strain relief 215 is provided. The subject to be treated receives an external controller comprising an communication system connected to control circuitry. The external control unit can be programmed for various signal parameters including options for frequency selection, pulse width, pulse amplitude, duty cycle, etc.

In some embodiment, the nerves AVN and/or PVN are indirectly stimulated by passing electrical signals through the tissue surrounding the nerves. In some embodiments, the electrodes are bipolar pairs (i.e. alternating anode and cathode electrodes). In some embodiments, a plurality of electrodes may be placed overlying the AVN and/or PVN. As a result, energizing the plurality of electrodes will result in application of a signal to the AVN and/or PVN and/or their branches. In some therapeutic applications, some of the electrodes may be connected to a upregulating electrical signal source (e.g., with a low frequency and other suitable parameters as described below) and other electrodes may apply a downregulating signal (e.g., with a high frequency and/or other suitable parameters as described below). In some embodiments, only a single array of electrodes could be used with all electrodes connected to a upregulating or a downregulating signal. In some therapeutic applications, some of the electrodes may be connected to an upregulating electrical signal source (with a suitable frequency and other parameters as described below).

In other embodiments, a plurality of electrodes are placed overlying the hepatic and/or celiac branches of the AVN and/or PVN nerves. In some therapeutic applications some of the electrodes may be connected to a upregulating electrical signal source (with a low frequency and other suitable parameters described below) and other electrodes may apply a downregulating signal. In some therapeutic application an electrode connected to a blocking electrical signal is placed on the hepatic branch of the vagal nerve. In other therapeutic applications an electrode connected to an upregulating signal is placed on the celiac branch of the vagal nerve. In still yet other therapeutic applications a first electrode connected to an upregulating signal is placed on the hepatic branch and a second electrode, connected to an downregulating signal is place on the celiac branch.

The electrical connection of the electrodes to an pulse generator may be as previously described by having a leads (e.g. 106,106a) connecting the electrodes directly to an implantable pulse generator (eg. 104). Alternatively and as previously described, electrodes may be connected to an implanted communication system for receiving a signal to energize the electrodes.

Two paired electrodes may connect to a pulse generator for bi-polar signal. In other embodiments, a portion of the vagal nerve is dissected away from the esophagus. An electrode is placed between the nerve and the esophagus. Another electrode is placed overlying the vagal nerve on a side of the nerve opposite the first electrode and with electrodes axially aligned (i.e., directly across from one another). Not shown for ease of illustration, the electrodes may be carried on a common carrier (e.g., a PTFE or silicone cuff) surrounding the nerve VN. Other possible placements of electrodes are described in US 2005/0131485, the disclosure of which is hereby incorporated by reference in its entirety.

While any of the foregoing electrodes could be flat metal pads (e.g., platinum), the electrodes can be configured for various purposes. In an embodiment, an electrode is carried on a patch. In other embodiments, the electrode is segmented into two portions both connected to a common lead and both connected to a common patch. In some embodiments, each electrode is connected to a lead and placed to deliver a therapy from one electrode to another. A flexible patch permits articulation of the portions of the electrodes to relieve stresses on the nerve.

The present system may contain software to permit use of the system 100 in a programmable variety of therapy schedules, electrical signal delivery, therapy programs, operational modes, system monitoring and interfaces as will be described herein. In embodiments, system software can be stored on a variety of computer devices, such as an external smartphone or tablet, external programmer, the neuroregulator, and/or external charger.

Now referring to FIG. 5, an exemplary architecture of a computing device that can be used to implement aspects of the present disclosure is illustrated. For example, the external charger 101, the neuroregulator 104, an external programmer, an external smartphone of tablet, or various systems and devices of the therapy system 100 can be implemented with at least some of the components of the computing device as illustrated in FIG. 5. Such a computing device is designated herein as reference numeral 300. The computing device 300 is used to execute the operating system, application programs, and software modules (including the software engines) described herein.

The computing device 300 includes, in some embodiments, at least one processing device 302, such as a central processing unit (CPU). A variety of processing devices are available from a variety of manufacturers, for example, Intel or Advanced Micro Devices. In this example, the computing device 300 also includes a system memory 304, and a system bus 306 that couples various system components including the system memory 304 to the processing device 302. The system bus 306 is one of any number of types of bus structures including a memory bus, or memory controller: a peripheral bus; and a local bus using any of a variety of bus architectures. Examples of computing devices suitable for the computing device 300 include a desktop computer, a laptop computer, a tablet computer, a mobile device (such as a smart phone, an iPod® mobile digital device, or other mobile devices), or other devices configured to process digital instructions.

The system memory 304 includes read only memory 308 and random access memory 310. A basic input/output system 312 containing the basic routines that act to transfer information within computing device 300, such as during start up, is typically stored in the read only memory 308.

The computing device 300 also includes a secondary storage device 314 in some embodiments, such as a hard disk drive, for storing digital data. The secondary storage device 314 is connected to the system bus 306 by a secondary storage interface 316. The secondary storage devices and their associated computer readable media provide nonvolatile storage of computer readable instructions (including application programs and program modules), data structures, and other data for the computing device 300.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 314 or memory 304, including an operating system 318, one or more application programs 320, other program modules 322, and program data 324.

In some embodiments, computing device 300 includes input devices to enable a user to provide inputs to the computing device 300. Examples of input devices 326 include a keyboard 328, pointer input device 330, microphone 332, and touch sensitive display 340. Other embodiments include other input devices 326. The input devices are often connected to the processing device 302 through an input/output interface 338 that is coupled to the system bus 306. These input devices 326 can be connected by any number of input/output interfaces, such as a parallel port, serial port, game port, or a universal serial bus. Wireless communication between input devices and interface 338 is possible as well, and includes infrared, BLUETOOTH® wireless technology, WiFi technology (802.11a/b/g/n etc.), cellular, or other radio frequency communication systems in some possible embodiments.

In this example embodiment, a touch sensitive display device 340 is also connected to the system bus 306 via an interface, such as a video adapter 342. The touch sensitive display device 340 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 340, the computing device 300 can include various other peripheral devices (not shown), such as speakers or a printer.

The computing device 300 further includes a communication device 346 configured to establish communication across the network. In some embodiments, when used in a local area networking environment or a wide area networking environment (such as the Internet), the computing device 300 is typically connected to the network through a network interface, such as a wireless network interface 348. Other possible embodiments use other wired and/or wireless communication devices. For example, some embodiments of the computing device 300 include an Ethernet network interface, or a modem for communicating across the network. In yet other embodiments, the communication device 346 is capable of short-range wireless communication. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The computing device 300 typically includes at least some form of computer-readable media. Computer readable media includes any available media that can be accessed by the computing device 300. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 300.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

As described above, the computing device typically includes at least some form of computer-readable media. Computer readable media includes any available media that can be accessed by the computing device. By way of example, computer-readable media include computer readable storage media and computer readable communication media.

The computer implemented methods as described herein are implemented by storing a series of instructions on the neuroregulator, external programmer, and/or the external charger. In embodiments, a user may select parameters of the electrical signal therapy and upon selection, selects a combination of electrical signal treatments for the therapy program(s).

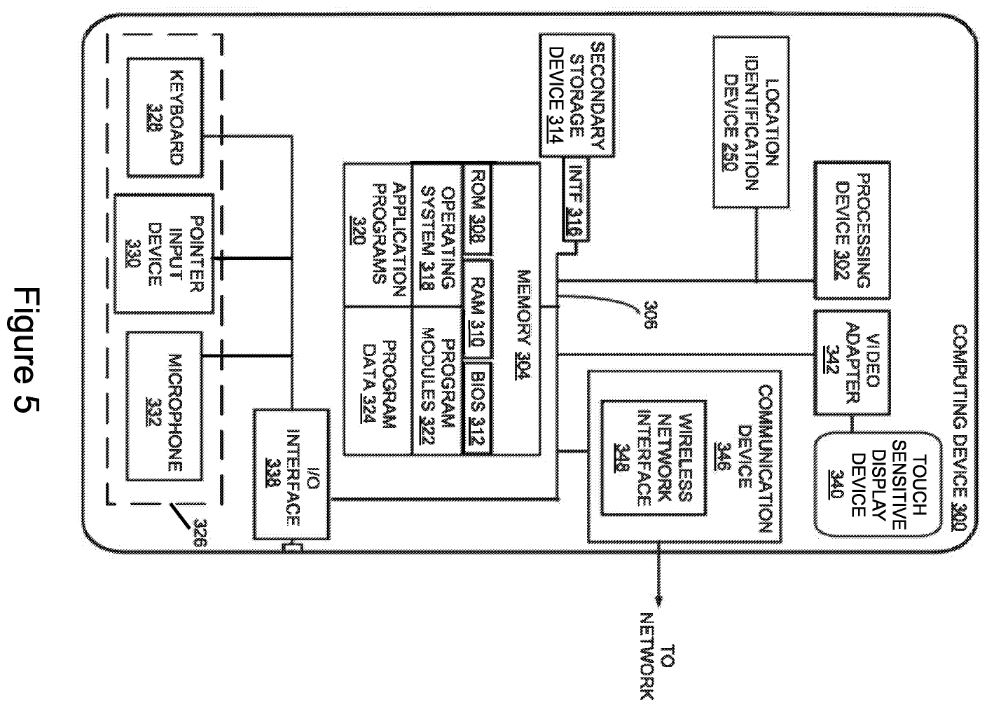

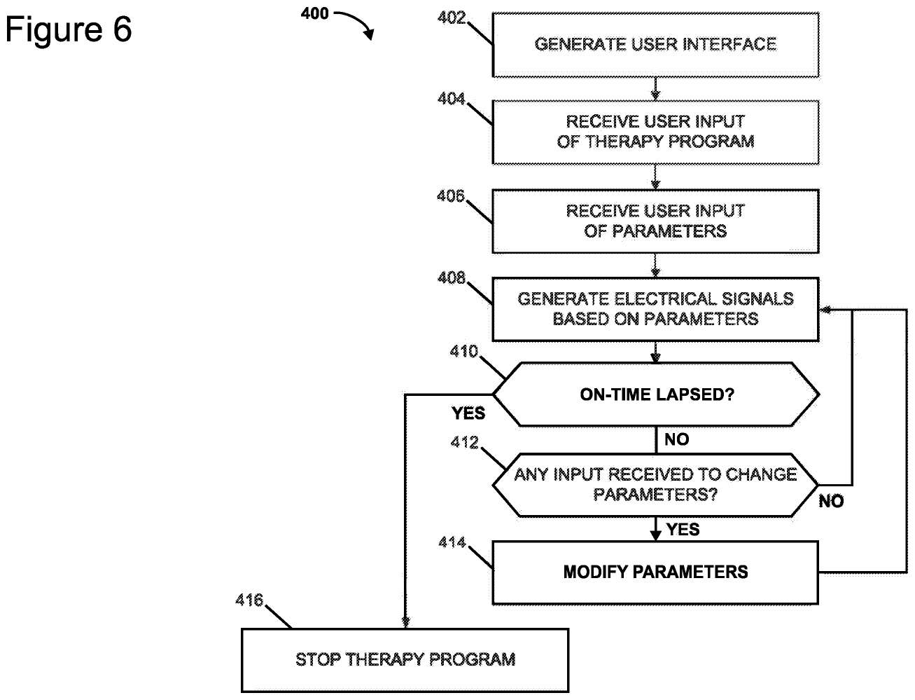

Now referring to FIG. 6, an example method 400 of operating the therapy system 100 is illustrated. At operation 402, the system 100 generates a user interface configured to receive various inputs from a user, such as one or more parameters, therapy programs, schedules, and any other information usable for system operation. At operation 404, the system 100 receives a user input of a therapy program via the user interface. As described herein, the system 100 is configured to provide a plurality of therapy programs, and the user can select one of the therapy programs available through the user interface. At operation 406, the system 100 receives a user input of one or more parameters that determine the characteristics of a therapy program.

At operation 408, the system 100 generates electrical signals based on the selected parameters, which implement the therapy program selected by the user. At operation 410, it is determined whether the on-time has lapsed. If so (“YES” at the operation 410), the system 100 stops the therapy program. If not (“NO” at the operation 410), the system 100 determines if there is any input for changing one or more of the parameters, at operation 412. If so (“YES” at the operation 412), the system 100 modifies the parameters based on the input, and continues the operation 408 and the subsequent operations. If not (“NO” at the operation 412), the system 100 continues the operation 408 and the subsequent operations.

As illustrated in FIG. 6, the system 100 receives and utilizes a plurality of parameters to generate various patterns of electrical signals for different therapy programs. Examples of the parameters are described as follows:

Parameters that are selected by a user include type of nerve or organ. In embodiments, the type of nerve is selected from vagal nerve, anterior vagus nerve, posterior vagus nerve, hiatus on posterior nerve, hepatic branch of vagal nerve, celiac branch of vagal nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.

In embodiments, a user can select parameters that feature a high frequency signal or a high frequency low duty cycle signal for downregulating/blocking nerve activity. A user can also select parameters that feature a low frequency stimulation signal for upregulating/stimulating nerve activity. A user can select parameters to independently and separately apply multiple electrical signals applied to multiple nerves or nerve branches/fibers. A user can also select parameters to concurrently or simultaneously apply multiple electrical signals applied to multiple nerves or nerve branches/fibers, or otherwise apply the multiple signals in a coordinated fashion.

Additional examples of the neuroregulator, pulse generator, electrode, biological sensor, therapy program, therapy schedule, electrical signal pattern, treatment parameters, etc., are described in US20210146136 and WO2020214982, the disclosure of which are hereby incorporated by reference in their entirety.

In some aspects, the present disclosure provides a method for making or assembling the system described herein for treating a condition associated with type-2 diabetes in a subject. In one example, a method comprises: (1) connecting a first electrode to an implantable pulse generator and placing the first electrode to a first nerve or organ: (2) optionally connecting a second electrode to the implantable pulse generator and placing the second electrode to a second nerve or organ: (3) configuring a programmable therapy delivery module of the implantable pulse generator to deliver at least one therapy program comprising a first electrical signal treatment and optionally a second electrical signal treatment, wherein the first electrical signal treatment is configured to be applied to the first nerve or organ through the first electrode, and the second electrical signal treatment is configured to be applied to the second nerve or organ through the second electrode, and wherein the first and/or the second electrical signal each initiate a neural stimulation or a neural block; and (4) configuring a programmable storage and communication module of an external component to store the at least one therapy program and to communicate the at least one therapy program to the implantable pulse generator.

In some embodiments, the first and/or the second electrical signal each have a frequency selected to initiate activity respectively on the first and/or the second target nerve or organ, and wherein the activity is an upregulation or downregulation of neural activity.

In some embodiments, the method further comprises connecting a glucose sensor to the implantable pulse generator; and monitoring or detecting plasma glucose level of the subject using the glucose sensor.

In some embodiments, the method further comprises configuring a communication system of the external component to communicate parameters of the at least one therapy program to an external communication device.

In some embodiments, the method further comprises connecting a transmitter to the glucose sensor to communicate data generated by the glucose sensor to an external communication device.

In some embodiments, the first and/or the second electrode are placed on the nerve or organ via a laparoscopic approach.

### Methods

In some aspects, the disclosure provides methods of treating a subject for a condition associated Type 1 and/or Type 2 diabetics.

It should be noted that type-2 diabetes may also be found where a patient has other diseases such as, but not limited to, kidney failure, certain tumors, liver disease, hypothyroidism, inborn errors of metabolism, severe infections, reactive hypoglycemia, and a number of drugs including alcohol use. The proposed device may help treat type-2 diabetes in patents with these medical conditions.

In some embodiments, a method comprises: applying an intermittent (or continuous) electrical signal to a target nerve or a target organ of a subject at a site with said electrical signal selected to upregulate and/or downregulate neural activity on the nerve or organ and with normal or baseline neural activity restoring upon discontinuance of said upregulation and/or downregulation. In embodiments, the method provides for an decrease in secretion of insulin and/or an increase in secretion of glucagon, or both. In some embodiments, the method provides for an increase in glucose concentration of the treated subject. In some embodiments, the methods further comprise administering a composition to the subject comprising an effective amount of an agent that increases glycemic control. In some embodiments, the electrical signal is applied to the nerve or organ by implanting a device or system as described herein.

In some embodiments, a method of treating a condition associated with type-2 diabetes in a subject in need thereof comprises applying an intermittent (or continuous) neural stimulation signal to a target nerve of the subject having a type-2 diabetes condition at a stimulating site with said neural stimulation signal selected to upregulate neural activity on the nerve and to restore neural activity on the nerve upon discontinuance of said stimulation.

In some embodiments methods include, treating a patient for type-2 diabetes with a concurrent treatment comprising: a) applying an intermittent (or continuous) neural stimulation signal to a target nerve or organ of the patient at multiple times per day and over multiple days with the stimulation signal selected to upregulate afferent and/or efferent neural activity on the nerve and with neural activity restoring upon discontinuance of said stimulation signal; and b) applying an intermittent (or continuous) neural block signal to a target nerve of the patient at multiple times per day and over multiple days with the stimulation selected to downregulate afferent and/or efferent neural activity on the nerve with neural activity restoring upon discontinuance of said block signal.

In some embodiments, a method of achieving glucose regulation in a patient comprises positioning an electrode on or near a vagal nerve branch, and an anodic electrode in contact with adjacent tissue: implanting a neurostimulator coupled to the electrodes into the patient, applying electrical pulses with defined characteristics of amplitude, pulse width, frequency and duty cycle to the vagal nerve branch wherein the defined characteristics are selected to improve glucose regulation or restoring the glucose level to a normal or desired level in the patient.

In some embodiments, a method comprises: applying an intermittent (or continuous) electrical signal to a target nerve or organ, with said electrical signal selected to upregulate or downregulate neural activity on the nerve or organ and to restore neural activity on the nerve upon discontinuance of said signal, wherein the electrical signal is selected to perform at least one of: increasing or modifying the amount of glucagon, decreasing or modifying the amount of insulin, or increasing the glucose level to reach or exceed a pre-determined level. In some embodiments, the electrical signal is selected for frequency, pulse width, amplitude, and timing to upregulate neural activity as described herein. In some embodiments, the electrical signal is selected for frequency, pulse width, amplitude and timing to downregulate neural activity as described herein. In some embodiments, the electrical signal is selected to increase or modify release of glucagon and/or to decrease or modify insulin by the pancreas, especially when plasma glucose is below a pre-determined threshold level. In some embodiments, the electrical signal is selected to modify liver sensitivity to glucagon.

In embodiments, the electrical signal is applied intermittently in a cycle including an on time of application of the signal followed by an off time during which the signal is not applied to the nerve, wherein the on and off times are applied multiple times per day over multiple days. In some embodiments, the on time is selected to have a duration of about 30 seconds to about 5 minutes. When the signal is selected to downregulate activity on the nerve, the electrical signal is applied at a frequency of about 200 Hz to about 10,000 Hz. When the signal is selected to upregulate activity on the nerve, the electrical signal is applied at a frequency of about 0.01 Hz up to about 200 Hz.

In embodiments, the electrical signal is applied to an electrode positioned on the vagal nerve. In some cases, the electrical signal is applied on the hepatic branch of the vagal nerve. In other cases, the electrical signal is applied on the celiac branch of the vagal nerve. In some embodiments, the electrical signal is applied to an organ involved in glucose regulation such as the liver, pancreas, duodenum, jejunum, or ileum.

In embodiments, downregulating and upregulating signals are both applied. In some cases, the signals are applied at the same time, different times, or overlapping times. In some embodiments, a downregulating signal is applied to a vagal nerve near the liver, and an upregulating signal is applied to a vagal nerve near the pancreas. In some embodiments, a downregulating signal is applied to the hepatic branch of the vagal nerve, and an upregulating signal is applied to the celiac branch of the vagal nerve.

In some embodiments, a method of treating a condition associated with type-2 diabetes in a subject comprises measuring plasma glucose levels following an intravenous (IV) glucose tolerance test (IVGTT) during stimulation of the celiac branch of the vagal nerve and with ligation, or high frequency alternating current (HFAC) blockade, of the vagal nerve hepatic branch.

In some embodiments, the method further comprises detecting the level of plasma glucose or glucagon or insulin to determine whether to apply an electrical signal treatment. If the levels of plasma glucose and/or glucagon are decreased to or below normal or baseline levels expected in a control sample from a subject having diabetes, treatment to increase glucagon and/or decreased insulin may by triggered until the plasma glucose levels rise to the expected levels required to maintain adequate control of the type-2 diabetes. Such levels are known or can be determined using methods known to those of skill in the art.

In some embodiments, the method further comprises administering an amount of an agent such as glucose, glucagon, or dextrose to facilitate the maintenance of type-2 diabetes.

In some embodiments, the method comprises applying a reversible intermittent (or continuous) modulating signal to a target nerve or organ of the subject in order to downregulate and/or upregulate neural activity on the nerve.

In some cases, the nerve is a nerve that innervates one or more alimentary organs, including but not limited to the vagal nerve, celiac nerves, hepatic branch of the vagal nerve, and splanchnic nerve. The signal applied may upregulate and/or downregulate neural activity on one or more of the nerves.

In some embodiments, said modulating signal comprises applying an electrical signal. The signal is selected to upregulate or downregulate neural activity and allow for restoration of the neural activity upon discontinuance of the modulating signal. A pulse generator, as described above, can be employed to regulate the application of the signal in order to alter the characteristic of the signal to provide a reversible intermittent (or continuous) signal. The characteristics of the signal include location of the signal, frequency of the signal, amplitude of the signal, pulse width of the signal, and the administration cycle of the signal. In some embodiments, the signal characteristics are selected to provide for treating a condition associated with type-2 diabetes.

In embodiments of the methods described herein a signal is applied to a target nerve at a site with said signal selected to upregulate neural activity on the nerve and with neural activity restoring upon discontinuance of said signal. In some embodiments, an upregulating signal may be applied to a first nerve or organ in combination with a down regulating signal applied to a second nerve or organ in order to improve glucose regulation.

The signal is selected to upregulate neural activity and allow for restoration of the neural activity upon discontinuance of the signal. A pulse generator, as described above, is employed to regulate the application of the signal in order to alter the characteristic of the signal to provide a reversible intermittent (or continuous) signal. The characteristics of the signal include frequency of the signal, location of the signal, and the administration cycle of the signal.

In some embodiments, electrodes applied to a target nerve are energized with an upregulating signal. The signal is applied for a limited time (e.g., 5 minutes). The speed of neural activity recovery varies from subject to subject. However, 20 minutes is a reasonable example of the time needed to recover to baseline. After recovery, application of an up signal again upregulates neural activity which can then recover after cessation of the signal. Renewed application of the signal can be applied before full recovery. For example, after a limited time period (e.g., 10 minutes) upregulating signal can be renewed.

In some embodiments, an upregulating signal may be applied in combination with a downregulating signal in order to improve glucose regulation, increase/modify the amount of secretion of glucagon, decrease/modify the amount of insulin, and/or increase the amount of plasma glucose. The neural regulation signals can influence the sensitivity to glucagon by the liver, the amount of glucose absorbed from food, and the amount of glucagon and/or insulin secreted from the pancreas. The neural regulation provides for a decrease in the amount of insulin required by the subject.

The upregulating and downregulating signals may be applied to different nerves at the same time, applied to the same nerve at different times, or applied to different nerves at different times. In embodiments, an upregulating signal may be applied to a celiac nerve or splanchnic nerve. In other embodiments, an upregulating or downregulating signal may be applied to a hepatic branch of the vagal nerve or the signal may be applied to increase or control the amount of glucose secreted from the liver.

In some embodiments, a upregulating signal is applied to a vagal nerve branch intermittently multiple times in a day and over multiple days in combination with an downregulating signal applied intermittently multiple times in a day and over multiple days to a different nerve or organ. In some embodiments, the upregulating signal is applied due to a sensed event such as the amount of plasma glucose present. In other embodiments, an upregulating signal applied to the splanchnic nerve or the celiac nerve can be applied during a time period after normal meal times for the subject typically 15 to 30 minutes after mealtimes or times when plasma glucose levels decrease.

In some cases, signals are applied at specific times. For example, a downregulating signal may be applied before and during meal, followed by a stimulatory signal about 30 to 90 minutes after eating. In another example, an upregulating signal may be applied to the vagal nerve or the celiac branch of the vagal nerve late in the evening when the glucose is decreasing.

In some embodiments, a stimulation signal is applied to the celiac branch of the vagal nerve when a monitor detects low plasma glucose levels. In other embodiments, a downregulating signal is continuously delivered to the hepatic branch of the vagal nerve, or the ventral vagal trunk above the branching point of the hepatic nerve, along with stimulation of the celiac branch, or the dorsal vagal trunk above the branching point of the celiac nerve. However, if an internal monitor detected plasma glucose reaching an undesirable hypoglycemic state the blocking signal would cease and stimulation would continue alone.

Modulation of neural activity can be achieved by upregulating and/or down regulating neural activity of one or more target nerves or organs.

In some embodiments, electrodes can be positioned at a number of different sites and locations on or near a target nerve. Target vagal nerve branches include the celiac nerve, the hepatic nerve, the vagal nerve, the splanchnic nerve, or some combination of these, respectively, of a subject. The electrode may also be positioned to apply a signal to an organ in proximity to the vagal nerve such as the liver, duodenum, jejunum, ileum, spleen, pancreas, esophagus, or stomach. In some embodiments, the electrode is positioned to apply an electrical signal to the nerve at a location distal to the diaphragm of the subject.

Electrodes may be positioned on different nerves to apply a downregulating signal as opposed to an upregulating signal. For example, a down regulating signal can be applied on the hepatic nerve and an upregulating signal applied to the celiac nerve. In some embodiments, the signals may be applied to reduce the neurally mediated reflex secretion by blocking the vagal nerves to the liver, and concurrently or subsequently, stimulate the celiac to inhibit insulin secretion and/or upregulate the celiac nerve to stimulate glucagon production.

In some embodiments, the electrode is positioned to apply a signal to a branch or trunk of the vagal nerve. In other embodiments, the electrode is positioned to apply a signal to a ventral trunk, dorsal trunk or both. In some embodiments, the electrodes may be positioned at two different locations at or near the same nerve or on the nerve and on an alimentary tract organ.

In some embodiments, a downregulating signal has a frequency of at least 200 Hz and up to 5000 Hz. In other embodiments, the signal is applied at a frequency of about 500 to 5000 Hz. In some embodiments, a downregulating signal has a frequency of 3,000 Hz to 5,000 Hz or greater when applied by two or more bi-polar electrodes. Such a signal has a preferred pulse width of 100 micro-seconds (associated with a frequency of 5,000 Hz). A short “off” time in the pulse cycle (e.g., between cycles or within a cycle) could be acceptable as long as it is short enough to avoid nerve repolarization. The waveform may be a square or sinusoidal waveform or other shape. The higher frequencies of 5,000 Hz or more have been found, in porcine studies, to result in more consistent neural conduction block. Preferably, the signal is bi-polar, bi-phasic delivered to two or more electrodes on a nerve.

In some embodiments, a signal amplitude of 0.01 to 20.0 mA is adequate for blocking. In other embodiments a signal amplitude of 0.01 to 10 mA is adequate for blocking. In still yet other embodiments a signal amplitude of 0.01 to 8 mA is adequate for blocking. Other amplitudes may suffice. Other signal attributes can be varied to reduce the likelihood of accommodation by the nerve or an organ. These include altering the power, waveform or pulse width.

Upregulating signals typically comprise signals of a frequency of less than 200 Hz, more preferably between 0.01 to 200 Hz, more preferably 10 to 50 Hz, more preferably 5 to 20 Hz, more preferably 5 to 10 Hz, more preferably 1 to 5 Hz, preferably 0.1 to 2 Hz, most preferably 1 Hz. Such a signal has a preferred pulse width of 0.1-10 microseconds. In some embodiments, a signal amplitude of 0.1 to 12 mA is adequate for stimulating. Other amplitudes may suffice. Other signal attributes can be varied to reduce the likelihood of accommodation by the nerve or an organ. These include altering the power, waveform or pulse width.

Selection of a signal that upregulates and/or downregulates neural activity and/or allows for recovery of neural activity can involve selecting signal type and timing of the application of the signal. For example, with an electrode conduction block, the block parameters (signal type and timing) can be altered by the pulse generator and can be coordinated with the stimulating signals. The precise signal to achieve blocking may vary from patient to patient and nerve site. The precise parameters can be individually tuned to achieve neural transmission blocking at the blocking site.

In some embodiments, the signal has a duty cycle including an ON time during which the signal is applied to the nerve followed by an OFF time during which the signal is not applied to the nerve. For example, the on time and off times may be adjusted to allow for partial recovery of the nerve. In some cases, the downregulating and upregulating signals can be coordinated so that the upregulating signals are applied when down regulating signals are not being applied such as when the upregulating signals are applied at specific times or due to sensed events. In some embodiments, a sensed event indicates that an upregulating signal is applied and a down regulating signal is not applied for a time period relating to the sensed event, e.g. plasma glucose is below a certain threshold. In preferred embodiments, the signal is continuously being applied.

In some embodiments, subjects receive an implantable component 104. (FIG. 3). The electrodes 212, 212a are placed on the AVN and/or PVN just below the patient's diaphragm. The external antenna (coil 102) (or other communication system) is placed on the patient's skin overlying the implanted receiving coil 105. The external control unit 101 can be programmed for various signal parameters including options for frequency selection, pulse amplitude and duty cycle. For stimulating signals, a frequency is selected of less than about 200 Hz. For blocking signals, the frequency options includes about 200 Hz to about 5,000 Hz. The amplitude options are 0-10 mA.

In some embodiments, an upregulating signal may be applied in combination with a down regulating signal in order to improve glucose regulation.

Normally a patient would only use the device while awake. The hours of therapy delivery can be programmed into the device by the clinician (e.g., automatically turns on at 7:00 AM and automatically turns off at 9:00 PM). In some cases, the hours of therapy would be modified to correspond to times when blood sugar fluctuates such as before a meal and 30-90 minutes after eating. For example, the hours of therapy may be adjusted to start at 5:00 AM before breakfast and end at 9:00 PM or later depending on when the last meal or snack is consumed. In the RF-powered version of the pulse generator, use of the device is subject to patient control. For example, a patient may elect to not wear the external antenna. The device keeps track of usage by noting times when the receiving antenna is coupled to the external antenna through radio-frequency (RF) coupling through the patient's skin.

In some embodiments, the external component 101 can interrogate the pulse generator component 104 for a variety of information. In some embodiments, therapy times of 30 seconds to 180 seconds per duty cycle are preferred to therapy times of less than 30 seconds per duty cycle or greater than 180 seconds per duty cycle.

During a 10 minute duty cycle (i.e., intended 5 minutes of therapy followed by a 5 minute OFF time), a patient can have multiple treatment initiations. For example, if, within any given 5-minute intended ON time, a patient experienced a 35-second ON time and 1.5 minute actual ON time (with the remainder of the 5-minute intended ON time being a period of no therapy due to signal interruption), the patient could have two actual treatment initiations even though only one was intended. The number of treatment initiations varies inversely with length of ON times experienced by a patient.

In some embodiments, a sensor may be employed. A sensing electrode SE can be added to monitor neural activity as a way to determine how to modulate the neural activity and the duty cycle. While sensing electrode can be an additional electrode to stimulating electrode, it will be appreciated a single electrode could perform both functions. The sensing and stimulating electrodes can be connected to a controller as shown in FIG. 3. Such a controller is the same as controller 102 previously described with the additive function of receiving a signal from sensing electrode.

In some embodiments, the sensor can be a sensing electrode, a glucose sensor, or sensor that senses other biological molecules or hormones of interest. When the sensing electrode SE yields a signal representing a targeted minimum vagal activity or tone, the controller with the additive function of receiving a signal from sensing electrode energizes the stimulating electrode BE with a upregulating signal. As described with reference to controller 102 (FIG. 3), controller with the additive function of receiving a signal from sensing electrode can be remotely programmed as to parameters of stimulation duration and no stimulation duration as well as targets for initiating an upregulating or downregulating signal.

Ideal model species for testing the current invention include Alloxan treated glucose intolerant swine, and to a lesser extent Zucker obese rats. These models are ideal given the similarity in anatomy with humans, and the similar pathology to type-2 diabetic patients. Swine have the additional advantage of being similar in scale to human anatomy, particularly as it relates to the Vagus nerve. Anatomy of the vagus pathways and associated organ systems are shown in FIG. 1. Alloxan treatment induces type-2 diabetes pathology in swine models. Swine were trained for 7 days prior to pre-implant oral glucose tests to wear a jacket to house 2 mobile chargers. During the charging sessions the swine were not restrained and there was no apparent stress to the animals. The future goal is the generation of a unitary implantable 5000 Hz/stimulation device. In swine experiments glycemic control was accessed by an oral glucose tolerance test during concurrent hepatic branch blockage and celiac branch stimulation. Insulin measurements were taken prior to and following swine experiments giving insight into beta cell exhaustion. In Zucker Obese rat experiments glycemic control was accessed with an intravenous glucose tolerance test during hepatic branch block with concurrent celiac stimulation.

The current invention showed increased glycemic control in animal models without significant tissue damage. Safety was evaluated using histopathology on the brain, liver, pancreas and nerve following the experiments. The current experiments suggest stimulation and block can be used in conjunction with continuous glucose monitoring devices to blunt glucose spikes & manage type 2 diabetes. There is opportunity for using stimulation and block as a closed loop system with AI & machine learning tools to optimize type 2 diabetes therapy. These studies demonstrate that the current invention is safe and shows efficacy by blunting of glucose spikes and accomplishing glycemic control.

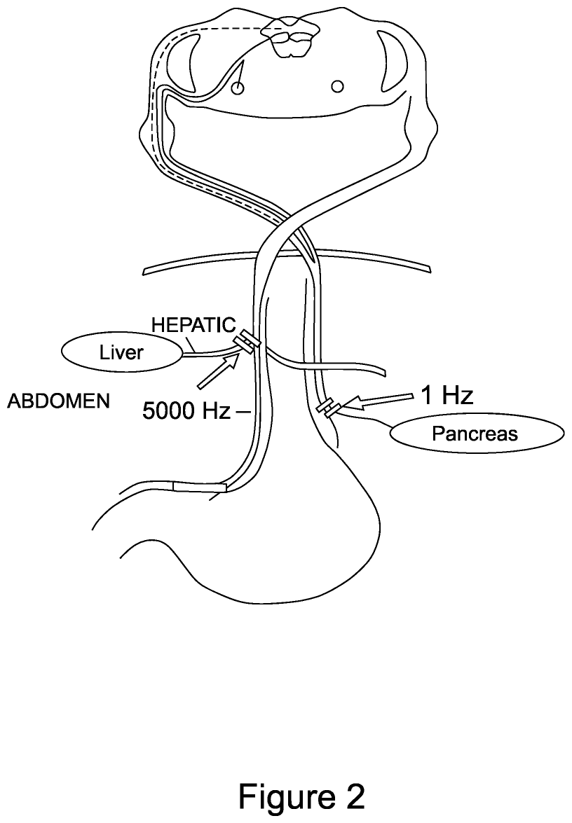

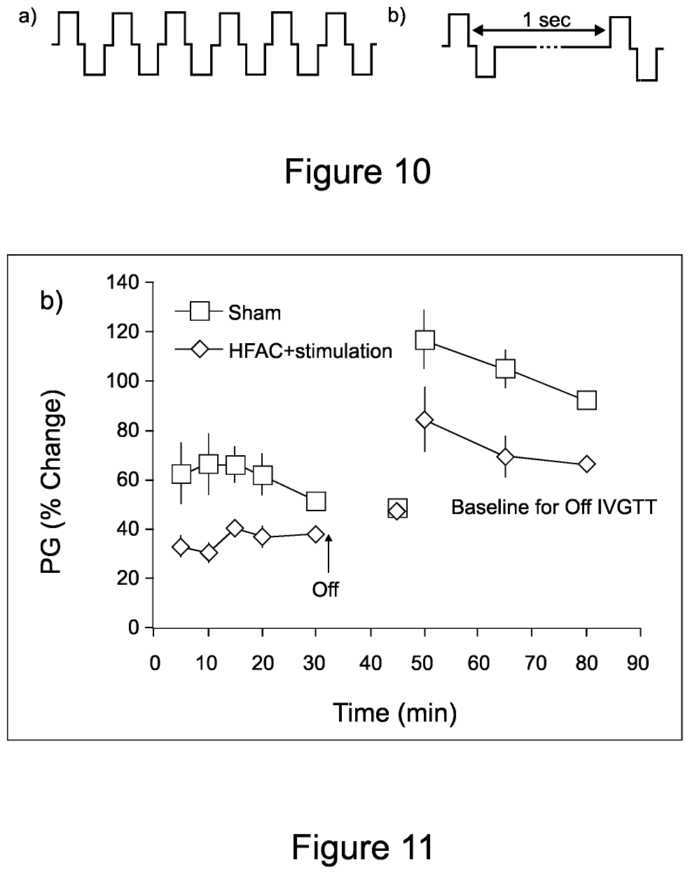

FIG. 1 diagrams the relevant pathways of the Vagus nerve below the diaphragm. The Vagus nerve extends from the brain stem (not pictured) and enters the abdominal cavity where it branches off to from the hepatic and celiac branches, among others. The hepatic branch enervates the liver, which in turn releases glucose into the blood stream. The disclosed treatment delivers electrical stimulation at a frequency of 5000 Hz to the hepatic branch, creating an electrical blockade and preventing stimulation of the liver. The celiac branch enervates the pancreas, stimulating the release of insulin from the pancreas. The disclosed treatment delivers electrical stimulation to the celiac branch at a frequency of 1 Hz, stimulating the pancreas. Block of the hepatic branches may decrease the livers sensitivity to glucagon and has been shown to decrease insulin resistance through attenuation of PPARα. Electrodes were anchored at the hepatic & celiac branching points. FIG. 2 illustrates the locations of the electrodes relative to the anatomical structures in the abdomen. Stimulation of the celiac branch has been shown to increases plasma insulin and glucagon. FIG. 10 shows the blocking 5000 Hz waveform applied to the hepatic branch as well as the stimulation 1 Hz waveform applied to the celiac branch of the vagus nerve.

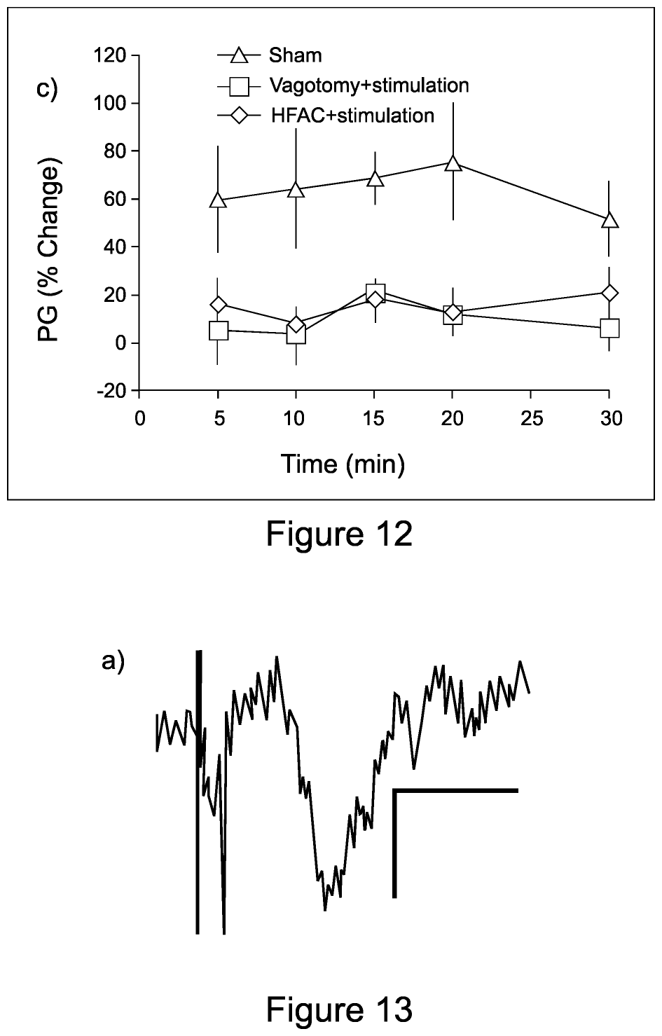

Tests in type-2 diabetic rat models involved the administration of intravenous glucose during experimental and control treatments. FIGS. 11 and 12 show the results of tests in rat models. FIG. 11 shows the average % change in Plasma glucose over time for rats in the experimental group and sham treatment group. Data taken between 0 and 30 minutes shows average % change in plasma glucose during treatments. Data take between 50 and 80 minutes shows average % change in plasma glucose 15 minutes following the cessation of treatments. Data at 45 minutes is the baseline blood glucose % change after cessation of intravenous glucose. As shown in FIG. 12 the average % change in Plasma glucose for rats in the experimental group compared to the sham treatment and vagotomy+stimulation treatment groups.

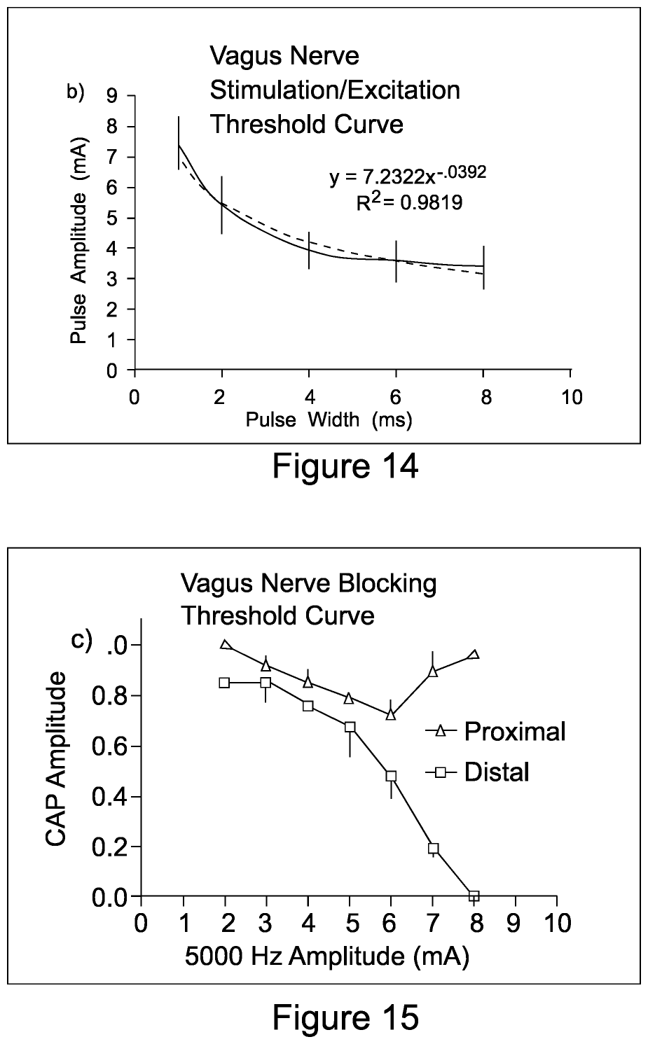

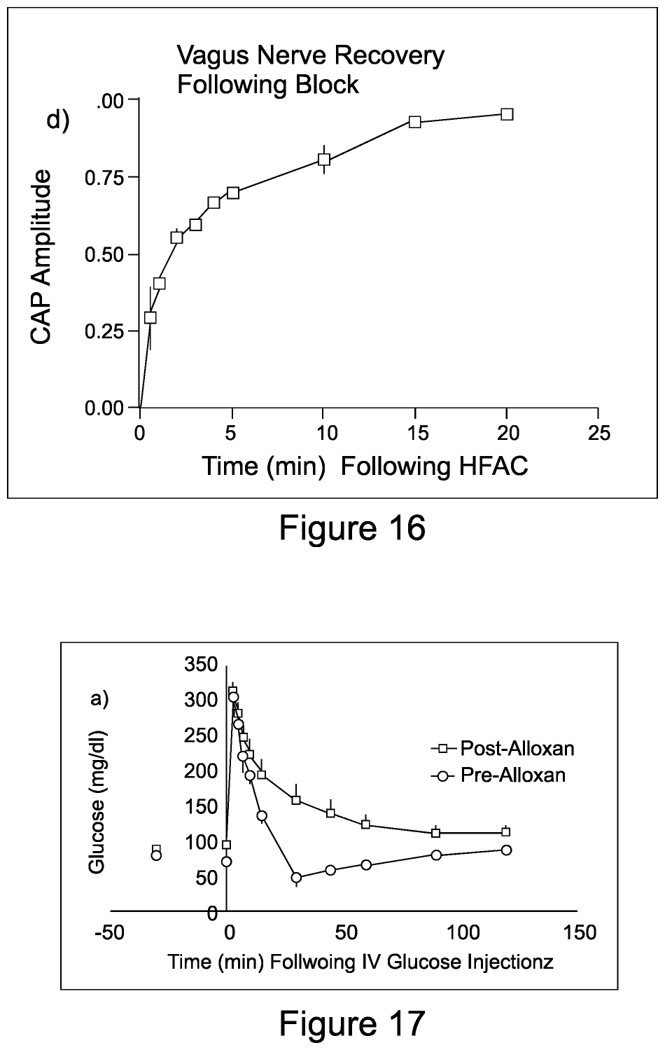

In preparation, isolated nerve electrophysiology was conducted to determine the electrical energy parameters to stim and block the swine sub-diaphragmatic vagal nerves. FIGS. 13-16 illustrate the assortment of tests which were conducted to establish these parameters. FIG. 13 illustrates the compound action potential was elicited when the nerve was stimulated. A strength duration curve was constructed to determine the excitability of the nerve and the stimulation parameters, which is shown in FIG. 14. Results from 5000 Hz blocking experiments, show in FIG. 15 demonstrated that 8 mA was the optimal current amplitude for block and that it took the nerve about 15 min to recover following block, as seen in FIG. 16. The same parameters used in the isolated swine vagus nerve experiments were used in the in vivo studies.

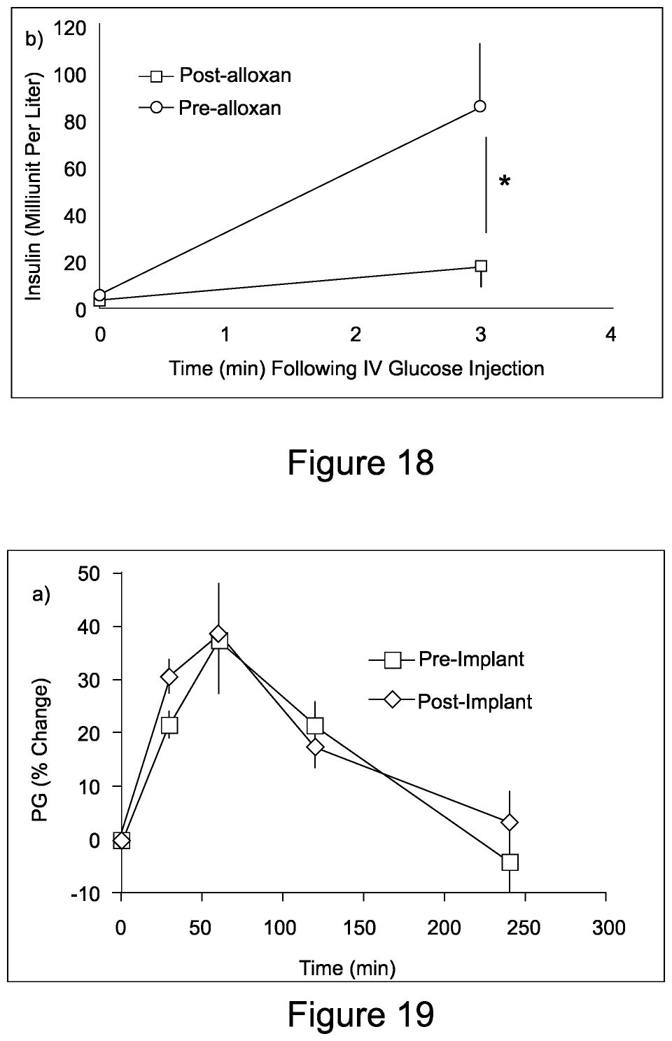

To ensure swine models would be appropriate for the testing the current invention, glucose tolerance and insulin response were tested. FIGS. 17 and 18 show the results of these tests and illustrate the appropriateness of the model for the current invention. The glucose curve shown in FIG. 17 demonstrates glucose intolerance, while the decreased insulin production seen in FIG. 18 shows appropriate reaction to alloxan. As such, swine were not insulin dependent and some pancreatic function was preserved demonstrating type 2 diabetes.

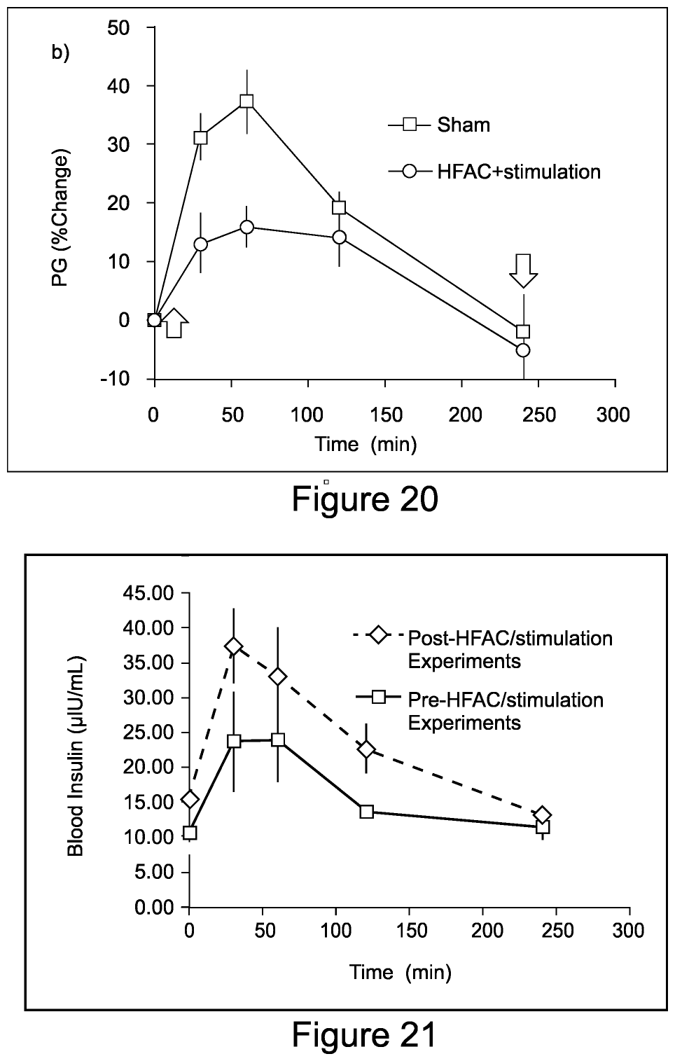

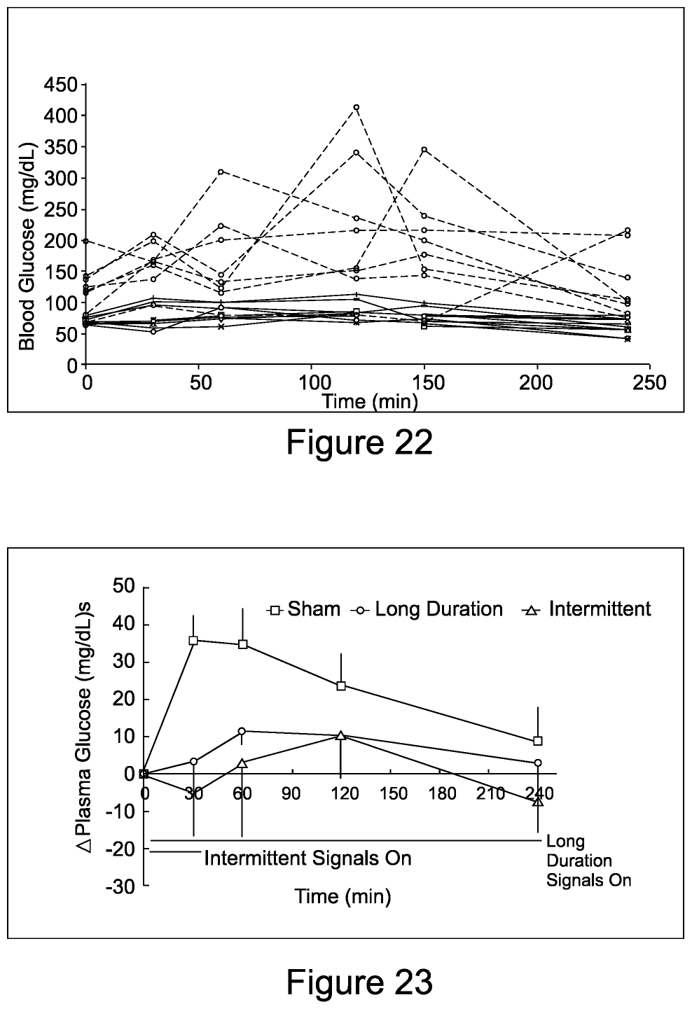

FIGS. 19 and 20 relate to tests conducted in type-2 diabetic swine models wherein glucose was administered orally. FIG. 19 shows the average % change in blood glucose levels over time in swine subjects before and after implant of electrodes. FIG. 20 shows the average % change in blood glucose levels over time in swine subjects receiving experimental or sham treatments. FIG. 21 which shows HFAC+stimulation in a Alloxan treated swine. A titrated dose of Alloxan-induced partial ablation of beta cells was utilized. Following Alloxan treatment, pigs had decreased glycemic control but were not insulin dependent. An IVGTT was conducted prior to, and following, the Alloxan treatment which demonstrated significantly increase in AUC following Alloxan (pre-Alloxan AUC=3237±362 AU, post-Alloxan AUC=7230±483 AU, p<0.001, students t-test). Following recovery from IVGTTs PG was 113±9. The anode and cathode electrodes delivering HFAC and stimulation were the same dimensions, configuration, separation and impedance (typically 1000 ohms) as used in related Vagus nerve electrophysiology study. FIG. 22 shows the fluctuation in blood glucose in swine subjects over the course of experimental versus sham treatment. FIG. 23 shows the average change in plasma glucose in swine subjects receiving intermittent experimental treatment (30 minute) compared to subjects receiving long duration experimental treatment (240) minutes) or sham treatment.

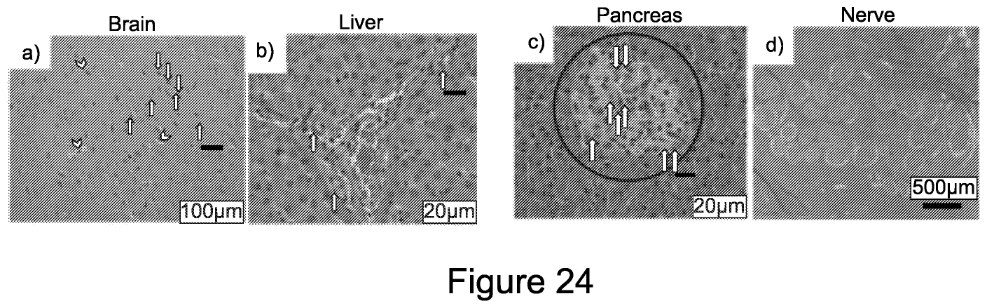

FIG. 24 shows the results of histopathology tests on the brain, liver, pancreas, and nervous tissue respectively, following the experimental treatment. The findings were normal in the brain, liver, pancreas, and nerves. Apoptosis in the islets of the pancreas was observed, which was consistent with alloxan treatment, and similar to the findings observed in the control group of alloxan treated swine that did not receive block and stimulation signals. Histopathology of brain, liver, pancreas tissues showed them to be healthy after the cessation of experiments. All Vagus nerve fibers and constituent axons within the cuff void were morphologically normal. There was no evidence of tissue damage at the cuffed electrode contact with the Vagus nerve.

### EXAMPLES

### Example 1—Implantable HVNS System

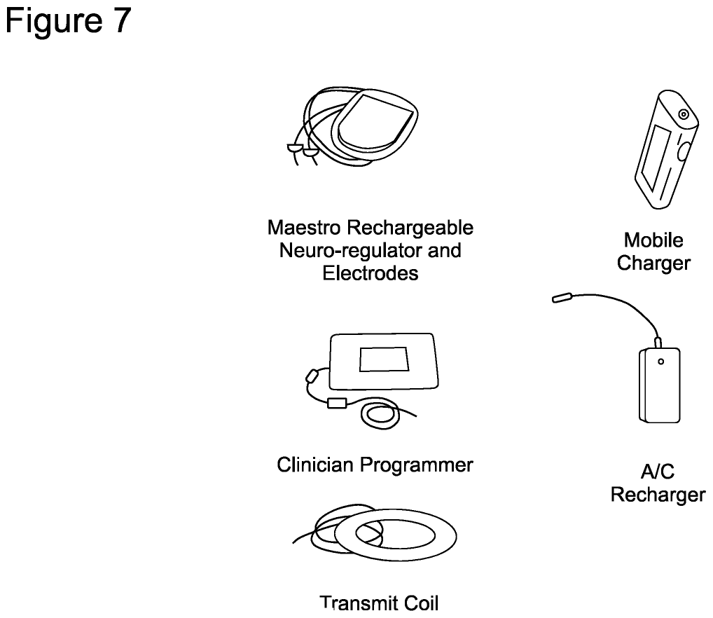

A particular example system is described in accordance with the present disclosure. FIG. 7 illustrates individual components of an example HVNS system. In the illustrated example, an implantable HVNS system comprises a Rechargeable Neuroregulator (RNR) pulse generator in combination with a Guardian™ Connect CGM system for glucose monitoring glucose. The HVNS system further includes two electrical leads with platinum-iridium electrodes which connect to the Rechargeable Neuroregulator (RNR) implantable pulse generator, a transmit coil, which is positioned over the RNR, outside the layer of the skin and communicates with the RNR through an antenna using a 6.73 MHz radio-frequency signal. The signal from the coil is used to charge the RNR as well as to program stimulation parameters. A mobile charger (MC) is connected to the transmit coil for charging and programming and a clinician programmer, which is connected to the MC for programming stimulation parameters. The Mobile Charger is recharged when connected to the AC Recharger. The Guardian™ Connect system includes a sensor inserted underneath the skin to measure glucose in the interstitial fluid. A transmitter is connected to the sensor and sends this information to the transmitter. The transmitter then wirelessly sends this data out to a smart device (e.g., iPhone or iPad) via blue-tooth technology, which displays plasma glucose levels.

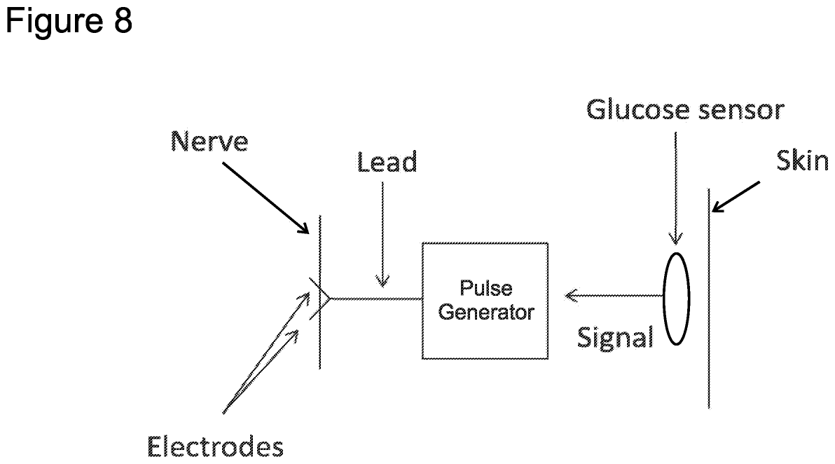

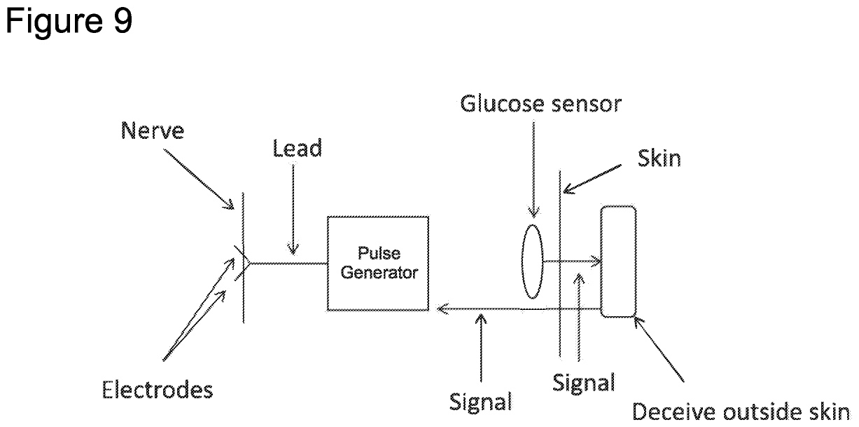

Referring to FIGS. 8-9, wherein the HVNS system would include a pulse generator, leads that are placed on the vagus nerve and an implantable glucose sensor (to monitor plasma glucose levels). The sensor sampling rate would be from about 1 second to 10 min. FIG. 8 shows a schematic of system in which an implantable glucose sensor communicates with a pulse generator to initiate vagus nerve stimulation. The implantable sensor would detect low plasma glucose levels and send a signal to turn the pulse generator on. FIG. 9 shows a schematic of system in which an implantable glucose sensor communicates first with an external device attached to the outside of the skin which then communicates with the pulse generator to initiate vagus nerve stimulation.

The communication between the pulse generator and the glucose sensor can be through, but not limited to, blue tooth technology, radio frequency, WIFI, light or sound. In some embodiments the glucose sensor would be below the layer of the skin and communicate to a device outside of the skin with a battery to power wireless communication. The communication between the glucose sensor and the device outside the body can be through, but not limited to, blue tooth technology, radio frequency, WIFI, light or sound. The device outside of the skin would then communicate with the pulse generator through, but not limited to, blue tooth technology, radio frequency, WIFI, light or sound. The implantable glucose sensor, or the external device that communicates with the implantable glucose sensor, could also communicate with a smart device (such as a phone running an app) to display plasma glucose levels and send an alarm when plasma glucose reaches an unsafe low level. The communication to the smart device can be through, but not limited to, blue tooth technology, radio frequency, WIFI, light or sound. Stimulation parameters include a frequency range between 0.01 Hz to 200 Hz, current or voltage amplitude range: 0.1 mA to 12 mA or 0.1 to 12 volts, pulse width range: 0.1 ms to 10 ms. Stimulation can be continuous or bursting with inter-burst intervals ranging from milliseconds, seconds to minuets.

Site of stimulation include any segment of the vagus nerve. This includes sub-diaphragmatic anterior or posterior vagus trunks and branches of the sub-diaphragmatic vagal trunks such as the celiac branch originating from the posterior vagus trunk, the accessory celiac branch, originating from the anterior vagus trunk or the hepatic branch, originating from the anterior vagus trunk. Sites of stimulation also include the anterior or posterior thoracic vagus, or the left or right cervical vagus. Any combination of vagus nerve stimulation sites is included.

In other embodiments, the HVNS system is entirely closed looped with the primary cell RNR incorporating blue-tooth capability to directly communicate with the glucose transmitter. Low duty cycle on demand stimulation may facilitate use of a small primary cell device without the need for recharging. The CGM transmitter may communicate with a smart device allowing physicians to optimize therapy parameters during a controlled type-2 diabetes trial.

### Example 2

To make comparisons between swine and rat models, changes in glucose were normalized to baseline glucose. In swine, baseline glucose was measured 10 minutes prior to oral glucose tolerance test, and in rats baseline glucose was measures 5 minutes prior to intravenous glucose tolerance tests. For each species four sets of control experiments were conducted in addition to the experimental test: a sham operation, a positive control vagotomy, stimulation alone, and vagotomy+stimulation. Data from the positive control vagotomy group and solo stimulation group have been precluded as there was no significant difference between the results of those treatments and the sham treatment. Results are shown in FIGS. 11-12 and 14-22. In our rat study, ligation of the hepatic branch did not increase glycemic control following the IVGTT. Anesthesia can affect PG. In the studies there was a 10 day period following surgery before OGTT experiments were conducted and post-implant OGTTs yielded similar responses to pre-implant OGTTs. It is unlikely that anesthesia had a significant impact on the results of this study. Since increased glycemic control was observed with application of block and stimulation 5 min following the initiation of oral glucose tolerance tests, gives us the inspiration of our methodology to work with continuous glucose monitoring technology to blunt glucose spikes on demand.

As shown in FIG. 11, initial plasma glucose in rats increased by an average of 63±12% 5 min following the glucose administration in the sham group and remained elevated for a half hour with a partial recovery. The area under the curve (AUC) for the sham group was equal to 1543±257. For the experimental group, initial % change in plasma glucose did not increase as sharply, and the AUC was significantly decreased for the sham group (AUC=898±68, p<0.05). After 30 minutes, treatment was stopped. Baseline % change in plasma glucose was measured 15 minutes after cessation of treatment, and intravenous glucose was administered once again without treatment. Plasma glucose in both sham and experimental treatments increased, followed by a slight decrease over the course of thirty minutes. Despite a slightly attenuated response in the experimental group, both groups demonstrated a similar trend, suggesting functional recovery following cessation of experimental treatment. This recovery gives the current invention an advantage over previous treatment methods as it allows for greater control of treatment by physicians, and reduced likelihood of developing resistance to treatment such as is seen in vagotomy treatments.

FIG. 12 shows the % change in plasma glucose in the experimental group compared to two control groups: sham treatment, and vagotomy+stimulation treatment. As can be seen, the difference in the experimental group and sham treatment group resembles that of the initial test shown in FIG. 11. More importantly, FIG. 12 shows similar activity in % change between the experimental treatment and vagotomy+stimulation treatment. This data supports the efficacy electrical blockade in the hepatic branch as comparable to that of a vagotomy, but with the aforementioned advantages of control, and decreased likelihood of resistance to treatment.

FIG. 19 shows no significant change in ability to process plasma glucose as a result of surgery, precluding the implant as a potential source of variation in swine subjects. FIG. 20 shows the % change in plasma glucose over the course of 240 minutes following administration of oral glucose in experimental and sham treatment groups. Average AUC for the sham treatment group was 6228±1293. The experimental treatment group saw a significant decrease in AUC compared to the sham treatment group 2225±825, p=0.015, demonstrating efficacy of the current invention not only in rat models, but also in swine. This is a promising development and suggests efficacy in human patients given the similar anatomy and physiology of the model species.

### Example 3

The experiments conducted in Example 2 demonstrated increased average glycemic control as a result of the current invention but did not take into consideration fluctuation in plasma glucose between test subjects. Experimental design was similar to that of swine tests in Example 2: in the experimental group, celiac fibers were stimulated at a frequency of 1 Hz with concurrent application of high frequency alternating current (5000 Hz) blockade to hepatic fibers. A sham treatment control was also used to compare results. The results of the experiment are shown in FIG. 21.

The sham treatment group saw a high level of fluctuation, with a standard deviation of 62±11 mg/dL and a % coefficient of variation of 38±6%. The experimental treatment group saw significantly reduced fluctuation, with a standard deviation of 13±1 mg/dL (p<0.01) and a % coefficient of variation of 17±2% (p<0.01). This finding is significant as large fluctuations in PG has been shown to increase oxidative stress and lead to co-morbidities in type-2 diabetes. This data demonstrates that the current invention not only leads to increased glycemic control, but also reduces potentially dangerous plasma glucose spikes.

### Example 4

In previous experiments block and stim was delivered for the full four hours of the glucose tolerance test, which used a considerable amount of energy. This prompted exploration intermittent block and stimulation to maintain glycemic control by applying the signals only at the first 30 min of the glucose tolerance test in alloxan induced type 2 diabetic swine. Glycemic control was assessed using an oral glucose tolerance test (OGTT) in Alloxan treated swine.

In previous experiments, concurrent stimulation and electrical blockade were applied for extended periods. To limit energy consumption, the efficacy of shorter treatment durations (30 minutes) was tested compared to long duration treatment (240 minutes). The results of the experiment are show in FIG. 22.

### Example 5

While the preceding examples proved the efficacy of the current invention, the question of safety is still a concern. Whereas traditional vagotomy causes significant and irreversible tissue damage, the current invention shows greatly reduced damage to tissues involved. Tissue damage was assessed via histopathology of tissue samples taken from swine subjects. Results are shown in FIG. 23.

Following the experiments safety was demonstrated using histopathology of the brain, liver, pancreas, and nerve. Arrowheads seen in the histopathology of the brain (Scale Bar: 100 μM) are increased clear space surrounding vasculature: arrows point to shrunken basophilic cells surrounded by clear space. These findings are common artifactual changes. In the liver (Scale Bar: 20 μM), mild biliary ductular hyperplasia is indicated by black arrows. Mild diffuse hydropic change is present, but not clinically relevant. The circle shown in the histopathology of the pancreas (Scale Bar: 20 μM), encloses a pancreatic islet. Black arrows indicate apoptotic cells similar to non-stimulated controls and expected following diabetic induction with alloxan-induced treatment. There was no evidence of tissue damage at the cuffed electrode contact with the Vagus nerve, in vagus nerve histopathology (Scale Bar: 500 μM). The host reaction to the cuff sheath & surgical fixation sites were typical for the animal model at the post-implantation time point. These findings are significant as it gives the current invention advantage over traditional vagotomy, which cause irreversible damage to the hepatic branch of the Vagus nerve and which loses potency over time as the patient develops resistance to treatment. By maintain healthy nerve tissue, the disclosed treatment can be utilized as necessary.

The following numbered clauses define further example aspects and features of the present disclosure:
1. A system for treating diabetes in a subject, the system comprising:
a first electrode adapted to be placed on and deliver electrical signal to a first nerve or organ:
optionally a second electrode adapted to be placed on and deliver electrical signal to a second nerve or organ:
an pulse generator operably connected to the first and/or the second electrode, wherein the pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and
an external component comprising a communication system and a programmable storage and communication module, wherein programmable storage and communication module are configured to store the at least one therapy program and to communicate the at least one therapy program to the pulse generator.
2. The system of clause 1, wherein the first and/or the second electrical signal are each independently configured to upregulate or down-regulate activity respectively on the first and/or second target nerve or organ.
3. The system of any one of clauses 1-2, wherein the first and the second electrical signals are applied concurrently, or simultaneously, or intermittently, or during substantially the same times, or during substantially different times, or in a coordinated fashion.
4. The system of any one of clauses 1-3, wherein the first and/or the second electrical signal treatments are each continuously applied to the first target nerve or organ and/or the second target nerve or organ respectively.
5. The system of any one of clauses 1-4, further comprising a glucose sensor configured to continuously monitor plasma glucose of the subject, wherein the glucose sensor is operably connected to the pulse generator and the external component.
6. The system of any one of clauses 1-5, wherein the glucose sensor is configured to detect an increase or decrease of plasma glucose from a pre-determined threshold level.
7. The system of clause 6, wherein the pulse generator is triggered to deliver the first and/or the second electrical treatment when the plasma glucose of the subject is of or below a first pre-determined threshold, and wherein the pulse generator ceases to deliver the first and/or the second electrical treatment when the plasma glucose of the subject is of or above a second pre-determined threshold.
8. The system of any one of clauses 1-7, wherein the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
9. The system of any one of clauses 1-8, wherein the first and/or the second electrical signals each have an on time and an off time, wherein the off time is selected to allow at least a partial recovery of the activity of the first and/or the second nerve or organ.
10. The system of clauses 9, wherein the on time is configured to commence upon the detection of plasma glucose level of 50 mg/dL and 90 mg/dL, or below 90 mg/dL, below 80 mg/dL, below 70 mg/dL, below 60 mg/dL, or below 50 mg/dL.
11. The system of any one of clauses 1-10, wherein the first and/or the second electrical signal treatment are configured to alter the plasma glucose level by at least about 5 mg/dL in about 10 minutes.
12. The system of any one of clauses 1-11, wherein the first and/or the second electrical signal treatment are configured to alter the plasma glucose level by at least about 10 mg/dL in about 20 minutes.
13. The system of any one of clauses 1-12, wherein the first and/or the second electrical signal treatment are configured to alter the plasma glucose level by at least about 20 mg/dL in about 30 minutes.
14. The system of any one of clauses 1-13, wherein the first electrical signal has a frequency of about 0.01 Hz to about 200 Hz.
15. The system of any one of clauses 1-13, wherein the first electrical signal has a frequency of about 200 Hz to about 10 k Hz.
16. The system of any one of clauses 1-15, wherein the second electrical signal has a frequency of about 0.01 Hz to about 200 Hz.
17. The system of any one of clauses 1-15, wherein the second electrical signal has a frequency of about 200 Hz to about 10 k Hz.
18. The system of any one of clauses 1-13, wherein the first electrical signal has a frequency of about 0.01 Hz to about 200 Hz, and wherein the second electrical signal has a frequency of about 200 Hz to about 10 k Hz.
19. The system of any one of clauses 1-18, wherein the first electrical signal and/or the second electrical signal each independently comprise a signal pattern, wherein each signal pattern comprises a pulse having a pulse width from about 0.1 microseconds to about 10,000 microseconds.
20. The system of any one of clauses 1-19, wherein the pulse of the first and/or the second electrical signal is monophasic pulse, or biphasic pulse, or combinations thereof.
21. The system of any one of clauses 1-20, wherein the first and/or the second electrical signal each independently have an on time of about 30 seconds to about 30 minutes.
22. The system of any one of clauses 1-21, wherein the first and/or the second electrical signal each independently have a current amplitude in a range from about 0.01 mAmps to about 20 mAmps.
23. The system of any one of clauses 1-22, wherein the first and/or the second electrical signal each independently comprise an abrupt start of pulses, or a ramp up of current/voltage amplitude, or a ramp up of frequency, or a ramping up of pulse widths, or combination thereof at or near initiation of applying the first and/or the second electrical signal.
24. The system of any one of clauses 1-23, wherein the first and/or the second electrical signal treatments are configured to be applied intermittently multiple times in a day and over multiple days, wherein the first and/or the second electrical signal each have a frequency selected to upregulate activity on the first nerve or organ and has an on time and an off time, wherein the off time is selected to allow at least a partial recovery of the activity of the first nerve or organ.
25. The system of any one of clauses 1-24, wherein the programmable storage and communication module are configured to store and communicate more than one therapy program, wherein each therapy program is different from one another, and is configured to be selected for communication.
26. The system of any one of clauses 1-25, further comprising a transmitter operably connected to the glucose sensor, wherein the transmitter is configured to communicate data generated by the glucose sensor to an external communication device.
27. The system of any one of clauses 1-26, wherein the communication system is selected from a group consisting of an antenna, blue tooth technology, radio frequency, WIFI, light, sound and combinations thereof, and wherein the communication system is configured to communicate parameters of the at least one therapy program to an external communication device.
28. A method of treating type-2 diabetes in a subject, the method comprising: applying a first electrical signal to a first nerve or organ of the subject using the system of clause 1, wherein the first electrical signal initiates a neural stimulation or a neural block; and optionally applying a second electrical signal to a second nerve or organ of the subject using the system of clause 1, wherein the second electrical signal initiates a neural stimulation or a neural block.
29. The method of clause 28, wherein the first and the second electrical signals are applied concurrently, or simultaneously, or intermittently, or during substantially the same times, or during substantially different times, or in a coordinated fashion.
30. The method of any one of clauses 28-29, wherein the first and/or the second electrical signal are configured to increase plasma glucose of the subject by at least about 5 mg/dL in about 10 minutes.
31. The method of any one of clauses 28-30, wherein the first and/or the second electrical signal treatment are configured to alter the plasma glucose level by at least about 10 mg/dL in about 20 minutes.
32. The system of any one of clauses 28-31, wherein the first and/or the second electrical signal treatment are configured to alter the plasma glucose level by at least about 20 mg/dL in about 30 minutes.
33. The method of any one of clauses 28-32, wherein the first and/or the second electrical signal are each applied continuously during an on time followed by an off time during which the signal is not applied to the nerve or organ.
34. The method of any one of clauses 28-33, wherein the on times are applied multiple times per day when plasma glucose level is about 50 mg/dL, about 60 mg/dL, about 70 mg/dL, or about 80 mg/dL.
35. The method of any one of clauses 28-34, wherein the off times are applied multiple times per day when plasma glucose level is at or above about 80 mg/dL to about 90 mg/dL, or above about 100 mg/dL, or above about 110 mg/dL.
36. The method of any one of clauses 28-35, wherein the first electrical signal has a frequency of about 0.01 Hz to about 200 Hz.
37. The method of any one of clauses 28-35, wherein the first electrical signal has a frequency of about 200 Hz to about 10 k Hz.
38. The method of any one of clauses 28-37, wherein the second electrical signal has a frequency of about 0.01 Hz to about 200 Hz.
39. The method of any one of clauses 28-37, wherein the second electrical signal has a frequency of about 200 Hz to about 10 k Hz.
40. The method of any one of clauses 28-35, wherein the first electrical signal has a frequency of about 0.01 Hz to about 200 Hz, and wherein the second electrical signal has a frequency of about 200 Hz to about 10 k Hz.
41. The method of any one of clauses 28-40, wherein the first nerve or organ and the second nerve or organ are independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
42. The method of any one of clauses 28-41, wherein the first nerve or organ and the second nerve or organ are different.
43. The method of any one of clauses 28-42, wherein the first electrical signal is applied on a hepatic branch of a vagus nerve or a ventral vagus nerve central to a branching point of a hepatic nerve.
44. The method of any one of clauses 28-42, wherein the first electrical signal is applied on a celiac branch of a vagus nerve or a ventral vagus nerve central to a branching point of a celiac nerve.
45. The method of any one of clauses 28-42, wherein the first electrical signal is applied to liver, pancreas or both.
46. The method of any one of clauses 28-45, wherein the second electrical signal is applied to a splanchnic nerve or a celiac branch of a vagus nerve, or pancreas.
47. The method of any one of clauses 28-45, wherein the second electrical signal is not involved in the method.
48. The method of any one of clauses 28-47, further comprising administering an agent that improves glucose control.
49. The method of clause 48, wherein the agent decreases the amount of insulin and/or decreases the sensitivity of cells to insulin.
50. A method of making a system for treating type-2 diabetes in a subject, the method comprising:
connecting a first electrode to an pulse generator and placing the first electrode to a first nerve or organ;
optionally connecting a second electrode to the pulse generator and placing the second electrode to a second nerve or organ;
configuring a programmable therapy delivery module of the pulse generator to deliver at least one therapy program comprising a first electrical signal treatment and optionally a second electrical signal treatment, wherein the first electrical signal treatment is configured to be applied to the first nerve or organ through the first electrode, and the second electrical signal treatment is configured to be applied to the second nerve or organ through the second electrode, and wherein the first and/or the second electrical signal each initiate a neural stimulation or a neural block; and configuring a programmable storage and communication module of an external component to store the at least one therapy program and to communicate the at least one therapy program to the pulse generator.
51. The method of clause 50, wherein the first and/or the second electrical signal each have a frequency selected to initiate activity respectively on the first and/or the second target nerve or organ, and wherein the activity is an upregulation or downregulation of neural activity.
52. The method of any one of clauses 50-51, further comprising connecting a glucose sensor to the pulse generator; and monitoring or detecting plasma glucose level of the subject using the glucose sensor.
53. The method of any one of clauses 50-52, further comprising configuring a communication system of the external component to communicate parameters of the at least one therapy program to an external communication device.
54. The method of any one of clauses 50-53, further comprising connecting a transmitter to the glucose sensor to communicate data generated by the glucose sensor to an external communication device.
55. The method of any one of clauses 50-54, wherein the first and/or the second electrode are placed on the nerve or organ via a laparoscopic approach.
56. A system for treating diabetes in a subject, the system comprising:
at least one electrode adapted to be placed on and deliver electrical signal to a nerve or organ of the subject:
an pulse generator operably connected to the at least one electrode, wherein the pulse generator comprises a power module and a programmable therapy delivery module, wherein the programmable therapy delivery module is configured to deliver at least one therapy program, wherein the at least one therapy program comprises at least one electrical signal treatment applied to the nerve or organ through the at least one electrode,
an external component comprising a communication system and a programmable storage and communication module, wherein programmable storage and
communication module are configured to store the at least one therapy program and to communicate the at least one therapy program to the pulse generator, and a glucose sensor operably connected and to and in communication with the pulse generator and the external component, wherein the glucose sensor is configured to continuously monitor plasma glucose of the subject and to detect an increase or decrease of plasma glucose from a pre-determined threshold level,
wherein, the pulse generator is triggered to deliver the at least one electrical signal treatment when the plasma glucose of the subject is of or below a first pre-determined threshold, and wherein the pulse generator ceases to deliver the at least one electrical signal treatment when the plasma glucose of the subject is of or above a second pre-determined threshold,
wherein the at least one electrical signal treatment is configured to initiate neural stimulation on the nerve or organ of the subject.
57. The system of clause 56, wherein the nerve or organ is selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
58. The system of any one of clauses 56-57, wherein the nerve is posterior vagus nerve (PVN) of the subject or the celiac vagus nerve branch of the PVN.
59. The system of any one of clauses 56-58, wherein the electrical signal has an on time and an off time, wherein the off time is selected to allow at least a partial recovery of the activity of the nerve or organ.
60. The system of clause 59, wherein the on time is configured to commence upon the detection of plasma glucose level of or below about 50 mg/dL, of or below about 60 mg/dL, of or below about 70 mg/dL, of or below about 80 mg/dL in the subject.
61. The system of any one of clauses 59-60, wherein the on time is about 30 seconds to about 30 minutes.
62. The system of any one of clauses 56-61, wherein the at least one electrical signal treatment comprises an electrical signal pattern having a frequency from about 1 Hz to about 200 Hz, or from about 1 Hz to about 50 Hz, or from about 1 Hz to about 20 Hz, or from about 1 Hz to about 10 Hz, or from about 1 Hz to about 5 Hz, or from about 1 Hz to about 2 Hz.
63. The system of any one of clauses 56-62, wherein the electrical signal pattern has a pulse width from about 0.1 microseconds to about 10 microseconds in about 0.1 microseconds steps.
64. The system of any one of clauses 56-63, wherein the electrical signal pattern has a pulse amplitude from about 0.1 mA to about 12 mA in about 0.1 mA steps.
65. The system of any one of clauses 56-64, wherein the pulse of the at least one electrical signal is monophasic pulse, or biphasic pulse, or combinations thereof.
66. The system of any one of clauses 56-65, wherein the at least one electrical signal further comprises an abrupt start of pulses, or a ramp up of current/voltage amplitude, or a ramp up of frequency, or a ramping up of pulse widths, or combination thereof at or near initiation of applying the electrical signal.
67. The system of any one of clauses 56-66, wherein at least one electrical signal treatment is configured to be applied intermittently multiple times in a day and over multiple days.
68. The system of any one of clauses 56-67, wherein the at least one electrical signal treatment is configured to causes increase of the plasma glucose of the subject by at least about 5 mg/dL in about 10 minutes, or by at least about 10 mg/dL in about 20 minutes, by at least about 20 mg/dL in about 30 minutes, or by at least about 30 mg/dL in about 45 minutes, or by at least about 40 mg/dL in about 60 minutes.
69. The system of any one of clauses 56-68, wherein the at least one electrical signal treatment is configured to cause an increase of insulin secretion in the subject.
70. The system of any one of clauses 56-69, wherein application of the at least one electrical signal treatment is configured to cause an decrease of insulin secretion in the subject.
71. The system of any one of clauses 56-70, further comprising a transmitter operably connected to the glucose sensor, wherein the transmitter is configured to communicate data generated by the glucose sensor to an external communication device.
72. The system of any one of clauses 56-71, wherein the communication system is selected from a group consisting of an antenna, blue tooth technology, radio frequency, WIFI, light, sound and combinations thereof, and wherein the communication system is configured to communicate parameters of the at least one therapy program to an external communication device.
73. A method of treating type-2 diabetes in a subject in need thereof, the method comprising:
applying the at least one electrical signal treatment to a posterior vagus nerve (PVN) of a subject or the celiac vagus nerve branch of the PVN of the subject using the system of any one of clauses 56-72.
74. The method of clause 73, wherein the electrical signal pattern applied to the has a frequency from about 1 Hz to about 20 Hz, a pulse width from about 0.1 microseconds to about 10 microseconds in about 0.1 microseconds steps, and a pulse amplitude from about 0.1 mA to about 12 mA in about 0.1 mA steps.
75. The method of any one of clauses 73-74, wherein application of the at least one electrical signal treatment causes increase of the plasma glucose of the subject by at least about 5 mg/dL, at least about 10 mg/dL, at least about 20 mg/dL, at least about 30 mg/dL, at least about 40 mg/dL, at least about 50 mg/dL, at least about 60 mg/dL, at least about 70 mg/dL, at least about 80 mg/dL, at least about 90 mg/dL, or at least about 100 mg/dL, in about 60 minutes.
76. The method of any one of clauses 73-75, wherein application of the at least one electrical signal treatment causes increase of insulin secretion in the subject.
77. The method of any one of clauses 73-76, wherein application of the at least one electrical signal treatment causes decrease of insulin secretion in the subject.
78. The method of any one of clauses 73-77, further comprising: placing the at least one electrode on the nerve or organ via a laparoscopic approach.

The above specification, examples and data provide a complete description of the manufacture and use of the composition of the invention. Since many embodiments of the invention can be made without departing from the spirit and scope of the invention, the invention resides in the claims hereinafter appended.

1. A system for treating diabetes in a subject, the system comprising:
a first electrode adapted to be placed on and deliver electrical signal to a first nerve or organ;
optionally a second electrode adapted to be placed on and deliver electrical signal to a second nerve or organ;
an pulse generator operably connected to the first and/or the second electrode, wherein the pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and
an external component comprising a communication system and a programmable storage and communication module, wherein programmable storage and communication module are configured to store the at least one therapy program and to communicate the at least one therapy program to the pulse generator.

2-27. (canceled)

28. A method of treating type-2 diabetes in a subject, the method comprising:
applying a first electrical signal to a first nerve or organ of the subject using the system of claim 1, wherein the first electrical signal initiates a neural stimulation or a neural block; and
optionally applying a second electrical signal to a second nerve or organ of the subject using the system of claim 1, wherein the second electrical signal initiates a neural stimulation or a neural block.

29. The method of claim 28, wherein the first and the second electrical signals are applied concurrently, or simultaneously, or intermittently, or during substantially the same times, or during substantially different times, or in a coordinated fashion.

30. The method of any one of claim 28, wherein the first and/or the second electrical signal are configured to increase plasma glucose of the subject by at least about 5 mg/dL in about 10 minutes.

31. The method of any one of claim 28, wherein the first and/or the second electrical signal treatment are configured to alter the plasma glucose level by at least about 10 mg/dL in about 20 minutes.

32. The system of any one of claim 28, wherein the first and/or the second electrical signal treatment are configured to alter the plasma glucose level by at least about 20 mg/dL in about 30 minutes.

33. The method of any one of claim 28, wherein the first and/or the second electrical signal are each applied continuously during an on time followed by an off time during which the signal is not applied to the nerve or organ.

34. The method of any one of claim 28, wherein the on times are applied multiple times per day when plasma glucose level is about 50 mg/dL, about 60 mg/dL, about 70 mg/dL, or about 80 mg/dL.

35. The method of any one of claim 28, wherein the off times are applied multiple times per day when plasma glucose level is at or above about 80 mg/dL to about 90 mg/dL, or above about 100 mg/dL, or above about 110 mg/dL.

36. The method of any one of claim 28, wherein the first electrical signal has a frequency of about 0.01 Hz to about 200 Hz.

37. The method of any one of claim 28, wherein the first electrical signal has a frequency of about 200 Hz to about 10 k Hz.

38. The method of any one of claim 28, wherein the second electrical signal has a frequency of about 0.01 Hz to about 200 Hz.

39. The method of any one of claim 28, wherein the second electrical signal has a frequency of about 200 Hz to about 10 k Hz.

40. The method of any one of claim 28, wherein the first electrical signal has a frequency of about 0.01 Hz to about 200 Hz, and wherein the second electrical signal has a frequency of about 200 Hz to about 10 k Hz.

41. The method of claim 28, wherein the first nerve or organ and the second nerve or organ are independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.

42.-55. (canceled)

56. A system for treating diabetes in a subject, the system comprising:
at least one electrode adapted to be placed on and deliver electrical signal to a nerve or organ of the subject;
an pulse generator operably connected to the at least one electrode, wherein the pulse generator comprises a power module and a programmable therapy delivery module, wherein the programmable therapy delivery module is configured to deliver at least one therapy program, wherein the at least one therapy program comprises at least one electrical signal treatment applied to the nerve or organ through the at least one electrode,
an external component comprising a communication system and a programmable storage and communication module, wherein programmable storage and communication module are configured to store the at least one therapy program and to communicate the at least one therapy program to the pulse generator, and
a glucose sensor operably connected and to and in communication with the pulse generator and the external component, wherein the glucose sensor is configured to continuously monitor plasma glucose of the subject and to detect an increase or decrease of plasma glucose from a pre-determined threshold level,
wherein, the pulse generator is triggered to deliver the at least one electrical signal treatment when the plasma glucose of the subject is of or below a first pre-determined threshold, and wherein the pulse generator ceases to deliver the at least one electrical signal treatment when the plasma glucose of the subject is of or above a second pre-determined threshold,
wherein the at least one electrical signal treatment is configured to initiate neural stimulation on the nerve or organ of the subject.

57.-72. (canceled)

73. A method of treating type-2 diabetes in a subject in need thereof, the method comprising:
applying the at least one electrical signal treatment to a posterior vagus nerve (PVN) of a subject or the celiac vagus nerve branch of the PVN of the subject using the system of claim 56.

74. The method of claim 73, wherein the electrical signal pattern applied to the has a frequency from about 1 Hz to about 20 Hz, a pulse width from about 0.1 microseconds to about 10 microseconds in about 0.1 microseconds steps, and a pulse amplitude from about 0.1 mA to about 12 mA in about 0.1 mA steps.

75. The method of claim 73, wherein application of the at least one electrical signal treatment causes increase of the plasma glucose of the subject by at least about 5 mg/dL, at least about 10 mg/dL, at least about 20 mg/dL, at least about 30 mg/dL, at least about 40 mg/dL, at least about 50 mg/dL, at least about 60 mg/dL, at least about 70 mg/dL, at least about 80 mg/dL, at least about 90 mg/dL, or at least about 100 mg/dL, in about 60 minutes.

76. The method of claim 73, wherein application of the at least one electrical signal treatment causes increase of insulin secretion in the subject.

77. The method of claim 73, wherein application of the at least one electrical signal treatment causes decrease of insulin secretion in the subject.

78. The method of claim 73, further comprising: placing the at least one electrode on the nerve or organ via a laparoscopic approach.
